(19) United States
(12) Patent Application Publication
(10) Pub. No.: US 2026/0185137 A1
(43) Pub. Date: Jul. 2, 2026

(54) FRET Active E2-UBL Conjugates and Uses Thereof

(71) Applicant: Board of Regents, The University of Texas System, Austin, TX (US)

(72) Inventors: Frank Herkules, San Antonio, TX (US); Dmitri Ivanov, San Antonio, TX (US)

(21) Appl. No.: 18/712,304

(22) PCT Filed: Oct. 28, 2022
(86) PCT No.: PCT/US2022/048268

§ 371 Date: May 22, 2024

Related U.S. Application Data
(60) Provisional application No. 63/282,213, filed on Nov. 23, 2021.

(51) Int. Cl. C12Q 1/48; C07K 14/47; G01N 33/542

(52) U.S. Cl. CPC C12Q 1/48; C07K 14/4702; G01N 33/542; C07K 2319/60; C07K 2319/95; G01N 2333/9108

(57) ABSTRACT

Certain embodiments are based on the discovery of a method for monitoring formation and dissociation of E2-Ubl conjugates in protein ubiquitination reactions. The methods use the fluorescence signal produced by a FRET-active E2-Ubl conjugate, wherein E2 is covalently labeled with one fluorophore, Ubl is labeled with a second fluorophore, and the two fluorophores form a FRET-active pair. The methods can be used in different assays in which E2-Ubl conjugates are formed or dissociated, for example in assays for monitoring enzymatic activity of E1 ubiquitin activating enzymes or enzymatic activity of E3 ubiquitin ligases. The method allows monitoring formation or/dissociation of E2-Ubl conjugates in real time with high sensitivity using widely available scientific instruments for measuring fluorescence and does not require labor intensive and error prone sample processing and analysis procedures. The method enables measurements of E1 or E3 activity in the high-throughput format.

### PRIORITY PARAGRAPH

This application is a 371 nationalization application of international application PCT/US2022/048268 filed Oct. 28, 2022 claiming priority to U.S. Provisional Patent Ser. No. 63/282,213 file Nov. 23, 2021, each of which is incorporated herein by reference in its entirety.

### STATEMENT REGARDING FEDERALLY FUNDED RESEARCH

This invention was made with government support under AI136697 awarded by the National Institutes of Health. The government has certain rights in the invention.

### REFERENCE TO SEQUENCE LISTING

A sequence listing required by 37 CFR 1.821-1.825 is being submitted electronically with this application. The sequence listing is incorporated herein by reference. The sequence listing that is contained in the file named “UTSKP0551USREV_seq” which is 18,391 bytes (as measured in Microsoft Windows®) and was created on 03/12/2026.

### BACKGROUND

Protein ubiquitination is an essential mechanism of the proteome's posttranslational regulation that contributes to virtually every facet of eukaryotic biology (Komander and Rape, Annu Rev Biochem 81, 203-29, 2012). Protein ubiquitination is the process of covalent attachment of an abundant cellular protein, ubiquitin (Ub), to another cellular protein, a ubiquitination substrate. This process is mediated by three distinct families of proteins: E1 ubiquitin activating enzymes (E1), E2 ubiquitin conjugating enzymes (E2), and E3 ubiquitin ligases (E3). Owing to the central role that protein ubiquitination plays in human biology, many members of these three protein families have emerged as promising therapeutic targets for various human diseases. Shortcomings of the existing biochemical assays for monitoring protein ubiquitination have posed significant obstacles for scientific progress and impeded discovery and development of inhibitors or activators of protein ubiquitination as therapeutic agents. This invention describes reagents and methods for robust detection and quantification of protein ubiquitination that will facilitate research and development of novel modulators of protein ubiquitination.

Protein ubiquitination normally involves two distinct steps. In the first step, the E1 ubiquitin activating enzyme uses the energy of ATP hydrolysis to synthesize a covalent conjugate of ubiquitin with an E2 ubiquitin conjugating enzyme. The E2-ubiquitin conjugate is the activated ubiquitin intermediate in the ubiquitination process, in which the C terminus of ubiquitin is covalently linked to an active site cysteine of the E2 via a high-energy thioester bond. In the second step, an E3 ubiquitin ligase catalyzes transfer of ubiquitin from the E2-ubiquitin conjugate onto an amine group of a substrate protein. In the ubiquitinated product the C-terminus of ubiquitin is covalently attached via an amide bond to either a lysine residue sidechain or the N-terminus of the substrate protein. The substrate protein could be a cellular protein recognized by the E3 ligase, another ubiquitin in the process of polyubiquitin chain synthesis, or the E3 ligase itself in an autoubiquitination process.

The protein ubiquitination process described above is a form of posttranslational modification of proteins that normally alters the function of the ubiquitinated substrate protein. The substrate protein is covalently modified with either a single ubiquitin unit (monoubiquitination), or with a linear or branched chain of multiple ubiquitin units linked to each other (polyubiquitination). The way multiple ubiquitin molecules are linked to each other in the polyubiquitin chain determines functional consequences of protein ubiquitination. The most well understood form of protein ubiquitination is the modification of the substrate protein with a linear polyubiquitin chain, in which the ubiquitin subunits are linked to each other via lysine 48 amino acid residue of the ubiquitin (K48-linked polyubiquitin). This form of polyubiquitination usually targets the polyubiquitinated substrate protein for proteasomal degradation.

In addition to ubiquitin there are other known posttranslational modifications that involve other small-proteins referred to as ubiquitin-like proteins (Ubls), or more specifically as type I ubiquitin-like proteins (Cappadocia and Lima, Chem Rev. 118(3):889-918, 2018) whose attachment process is very similar to that of ubiquitin and usually involves a similar sequence of events mediated by E1-like, E2-like and E3-like proteins. The Ubl proteins include, but are not limited to: SUMO1-4, NEDD8, ATG8, ATG12, URM1, UFM1, FAT10, and ISG15. Methods and reagents described here can be used to investigate protein modification with Ub or any of the Ubls that involves a E2-Ub/Ubl covalent intermediate.

The human genome encodes 8 E1-like proteins, more than two dozen of E2-like proteins and more than 600 E3 ubiquitin ligases. This hierarchy of the ubiquitination cascade illustrates that the versatility and richness of ubiquitin-mediated regulation are primarily controlled at the level of E3 ubiquitin ligases (Berndsen and Wolberger, Nat StructMol Biol 21, 301-7, 2014). Understanding how different E3 ubiquitin ligases select their substrates and how E3 activity is regulated is a rapidly evolving area of biomedical research.

One of the key measurements in the studies of bio-catalysis (catalysis of chemical reactions by macromolecular catalysts of biological origin called enzymes) is the ability to determine the rates of the chemical reactions catalyzed by enzymes. Performing rate measurements of protein ubiquitination is notoriously difficult. Most substrates, intermediates and products of the chemical reactions involved in the ubiquitination process are proteins, so the conversion of substrates to intermediates to products is usually monitored using SDS-PAGE gel electrophoresis. Samples are incubated for different periods of time and/or with different composition and/or concentration of reagents, the reactions are stopped by denaturation or heat inactivation and then analyzed by separating protein substrates, intermediates and products by mass using SDS-PAGE. Protein bands on the gel are then visualized and quantified using either a non-specific protein-binding dye (e.g., coomassie brilliant blue), or by Western Blot (WB) using antibodies that recognize a specific protein. SDS-PAGE monitoring of catalytic activity is cumbersome, time consuming, prone to sensitivity and variability issues and yields poor kinetic data. Furthermore, SDS-PAGE monitoring cannot be implemented in the high-throughput format, which is a severe obstacle for discovery of inhibitors of E3 ubiquitin ligases using high-throughput screening of compound libraries.

Another complication of measuring catalytic rates of protein ubiquitination is the multistep nature of the ubiquitination process. In-vitro ubiquitination reactions usually contain E1, E2, E3 proteins, ubiquitination substrate, ATP and numerous other reagents. As a consequence, it is difficult to verify what step of this multistep process is rate limiting in any particular composition of the ubiquitination reaction. The ubiquitin discharge assay was developed to partially overcome this problem. In the ubiquitin discharge assay, the E2-ubiquitin conjugate is first synthesized in the presence of E1 and ATP and the reaction is then stopped, usually by depleting ATP. The E2-ubiquitin conjugate is therefore formed prior to the addition of the E3 ligase to the reaction. Once the E3 ligase is added to the reaction, the rate of discharge of ubiquitin from the E2-ubiquitin conjugate is measured by collecting time points and quantifying the amount of the residual E2-ubiquitin conjugate by SDS-PAGE as described in the previous paragraph. The ubiquitin discharge assay is presently the most reliable tool for quantifying the catalytic activity of E3 ubiquitin ligases, but it still suffers from the limitations associated with SDS-PAGE detection, which are described in the previous paragraph.

In summary, there remains a need for additional compositions, methods, and assays for monitoring biochemical reactions involved in protein ubiquitination.

### SUMMARY

The Inventors provide a solution to the problems associated with current ubiquitination assays, e.g., the need for time consuming and error-prone SDS-PAGE analysis and protein quantification by Western blotting or nonspecific protein-binding dye. The solution is a fluorescent proximity assay that monitors changes in the intensity of fluorescence emitted by a FRET-active E2-Ub/Ubl conjugate. FRET-active E2-Ub/Ubl conjugates are prepared from Ub or Ubl covalently labeled with one fluorophore and E2 covalently labeled with another fluorophore. The two fluorophores form a FRET active pair, e.g., fluorescence donor and fluorescence acceptor, or fluorescence donor and fluorescence quencher. When the FRET-active E2-Ub/Ubl conjugate is synthesized in a reaction catalyzed by an E1 ubiquitin activating enzyme or when it is dissociated in a reaction catalyzed by an E3 ubiquitin ligase there is a change in the fluorescence intensity (increase or decrease) that is observed and can be directly measured. Embodiments of the invention are directed to FRET-active E2-Ub/Ubl conjugates, E2 proteins fluorescently labeled such that they can be used to synthesize FRET-active E2-Ub/Ubl conjugates and methods of using the FRET-active E2-Ub/Ubl conjugates and fluorescently labeled E2 proteins in various assays.

FRET refers to Fluorescence Resonance Energy Transfer, also termed Forster Resonance Energy Transfer. FRET is a phenomenon of excitation energy transfer from a fluorescent molecule excited by incident light (donor: energy donor, fluorescence donor) to a second fluorescent molecule(s) in the proximity of the donor (acceptor: energy acceptor, fluorescence acceptor). The transferred excitation energy can either be emitted as light by the acceptor or can be directly converted to heat in a non-radiative energy dissipation process. The latter process is usually described as fluorescence quenching and the energy acceptors that favor such non-radiative energy dissipation are called fluorescence quenchers.

FRET-active complex or molecule refers to a molecular entity, in which two fluorescent dyes (donor and acceptor) come into proximity to each other, such that the donor excitation energy can be transferred to the acceptor in a FRET process. The proximity of donor and acceptor can result from covalent linking of the donor and acceptor to the same molecule, or, alternatively, they can be covalently linked to two different molecules that associate with each other either covalently or non-covalently. The association of the molecules, to which the donor and acceptor are linked, can be either direct or indirect, e.g., when these two molecules form part of a larger multicomponent molecular complex.

The term proximity as used herein refers to a distance between the donor and acceptor of 5, 10, 20, 30, 40, 50, 60, 70, 80, 90, 100, up to 200 angstroms, including all values and ranges there between.

When the FRET acceptor emits the energy transferred from the donor as light, the wavelength of light emitted by the acceptor is longer than the wavelength of light emitted by the donor in the absence of FRET. FRET signal refers to the fluorescence signal observed when the excitation filter of the fluorimeter is selected to maximize excitation of the donor, whereas the emission filter of the fluorimeter is selected to maximize the signal emitted by the acceptor. When the donor comes into proximity with the acceptor in a FRET-active complex, the intensity of the FRET signal is increased because some of the donor excitation energy is transferred to the acceptor thus increasing the amount of light emitted by the acceptor.

When the FRET acceptor is a quencher, the energy is dissipated non-radiatively and no light is emitted by the acceptor. In this situation, fluorescence quenching is observed. Fluorescence quenching of the donor is detected with excitation and emission filters selected to maximize the fluorescence signal of the donor. When the donor comes into proximity with the quencher in a FRET-active complex, the intensity of donor fluorescence is decreased because excitation energy is transferred to the quencher and converted into heat.

FRET effect can also be used in time-resolved fluorescence energy transfer (TR-FRET) experiments. In time resolved experiments the excitation light is not applied to the sample continuously, but rather in bursts of certain duration (e.g., by using pulsed lasers for sample irradiation). The emission light is also not detected continuously but following a defined time delay after the excitation light pulse. TR-FRET experiments usually use a donor fluorophore with a long fluorescence lifetime (e.g., lanthanide ion complexes, such as Ln(III) chelates or cryptates) and an acceptor fluorophore whose excitation spectrum has a good overlap with the emission spectrum on the long-lifetime donor. The advantage of TR-FRET experiments is that they minimize the background signal arising from direct excitation of the acceptor by the excitation light. This increases the signal-to-noise of the FRET measurements.

Certain embodiments are directed to one or more reagents and/or components. One aspect is directed to a FRET-active E2-Ub/Ubl conjugate. This conjugate comprises one fluorescent entity (fluorescence donor dye, fluorescence acceptor dye or fluorescence quencher) covalently linked to the E2 portion of the E2-Ub/Ubl conjugate and a second fluorescent entity (fluorescence donor dye, fluorescence acceptor dye or fluorescence quencher) covalently linked to the Ub/Ubl component of the E2-Ub/Ubl conjugate. The fluorescent entities are configured such that when the E2 component is covalently coupled to the Ub/Ubl component such that the C-terminal carboxylate of the Ub/Ubl component forms a thioester bond with the active site cysteine of the E2 component the first and second fluorescent entities come into proximity and a FRET-active complex is formed. One aspect of the invention is the monitoring of the association or dissociation of the E2-Ub/Ubl conjugate by detecting a change (positive or negative) in the signal produced by association or disassociation of the FRET-active complex.

The association or formation of an E2-Ub/Ubl conjugate can be catalyzed by an E1 ubiquitin activating enzyme. Monitoring fluorescence signal of the FRET-active E2-Ub/Ubl conjugate whose formation is catalyzed by E1 offers a method to quantify catalytic activity of the E1.

The dissociation of an E2-Ub/Ubl conjugate can be catalyzed by E3 ligase. Monitoring fluorescence signal of the FRET-active E2-Ub/Ubl conjugate whose dissociation is catalyzed by E3 offers a method to quantify catalytic activity of the E3.

The methods allow for continuous, real-time monitoring of the E2-Ub/Ubl conjugates using a fluorimeter and bypassing the multiple steps (taking from hours to days) that are typically performed to process multiple time-point samples and to detect and quantify E2-Ub/Ubl conjugates or any other substrates, intermediates or products of the ubiquitination reaction using SDS-PAGE.

Another advantage of the FRET-active E2-Ub/Ubl conjugate method over traditional methods is its sensitivity and signal-to-noise characteristics. Fluorescence intensity can be measured with high signal-to-noise when the FRET-active E2-Ub/Ubl conjugates are present at nanomolar concentrations. This makes it possible to perform ubiquitination reactions at physiological concentrations of reagents, which is not the case when Coomassie brilliant blue stain is used to visualize substrates, intermediates, or products on SDS-PAGE.

Another advantage of the method is that it can be performed in a high-throughput format. Fluorescence signal detection can be performed using standard and widely available fluorescence plate readers, so the method allows analysis of tens, hundreds and even thousands of samples simultaneously. As such, the method will greatly facilitate discovery of bioactive compounds in large compound libraries using high-throughput screening (HTS). The method can enable HTS campaigns aimed at discovering inhibitors or activators of E1-like ubiquitin activating enzymes, inhibitors, or activators of E3 ubiquitin ligases, compounds for targeted protein degradation (TPDs), also known as proteasomal targeting chimeras (PROTACs), or molecular glues.

Certain embodiments are directed to methods for monitoring or assessing E3 ubiquitin (Ub) ligase activity. In certain aspects the methods include contacting a FRET-active E2-Ub/Ubl conjugate under conditions that form a FRET-active E2-Ub/Ubl conjugate with a sample to be monitored or assessed, and detecting a signal produced by an E2-Ub/Ubl conjugate wherein the signal will change over time in the presence of polypeptide having a E3 ubiquitin ligase activity, the ligase activity resulting in the dissociation of the E2-Ub/Ubl conjugate and the production of signal (increase or decrease in fluorescence). The Ub or Ubl molecule is labeled with a first fluorophore and the E2 molecule is labeled with a second fluorophore, the first fluorophore and second fluorophore forming a fluorescent resonance energy transfer (FRET) pair or a quenched pair. In certain aspects the FRET pair is selected from Alexa Fluor® 350, Alexa Fluor® 488, Alexa 25 Fluor® 532, Alexa Fluor® 546, Alexa Fluor® 568, Alexa Fluor® 594, Alexa Fluor® 647, or other know fluorphores. In certain aspects the E2 protein is selected from a protein encoded by E2 ubiquitin conjugating enzyme gene UBE2A, UBE2B, UBE2C, UBE2D1, UBE2D2, UBE2D3, UBE2E1, UBE2E2, UBE2E3, UBE2F, UBE2G1, UBE2G2, UBE2H, UBE2I, UBE2J1, UBE2J2, UBE2K, UBE2L3, UBE2L6, UBE2M, UBE2N, UBE20, UBE2Q1, UBE2Q2, UBE2R1, UBE2R2, UBE2S, UBE2T, UBE2U, UBE2V1, UBE2V2, UBE2Z, ATG2, BIRC5, or UFC. The ubiquitin moiety (Ub/Ubl) can be a ubiquitin or a ubiquitin-like peptide or protein. The Ub/Ubl molecule can be a ubiquitin having an amino acid sequence that is 95% identical to SEQ ID NO:1. In particular examples, the E2-Ub/Ubl pair comprise an Alexa Fluor 488 and an Alexa Fluor 594 FRET pair, or other known FRET pairs.

Certain embodiments are directed to a E2-Ub/Ubl conjugate covalently labeled with a FRET pair or quencher pair. In certain aspects the FRET pair are Alexa Fluor labels. In a particular aspect the Alexa Fluor labels are a 488/594 pair.

The abbreviations “Ub” and “Ubl” are used interchangeably throughout this document and refer to either ubiquitin or ubiquitin-like proteins. The term Ub/Ubl is used herein and refers to either ubiquitin or any other ubiquitin-like protein, unless a particular protein is referenced.

The term “E2” as used herein refers to any E2 ubiquitin conjugating enzyme that forms thioester-linked conjugates with any Ub/Ubl.

A “biological sample” in terms of the invention means a sample of biological tissue or fluid. Examples of biological samples are sections of tissues, blood, blood fractions, plasma, serum, urine, or samples from other peripheral sources; or cell cultures, cell colonies of even single cells, or a collection of single cells, including there lysates and cellular fractions as well as isolated recombinant proteins. Furthermore, also pools or mixture of the above mentioned samples may be employed. A biological sample may be provided by removing a sample of cells from a subject, but can also be provided by using a previously isolated sample. For example, a tissue sample can be removed from a subject suspected of having a disease by conventional biopsy techniques. In a preferred embodiment, a blood sample is taken from the subject. In one embodiment, the blood or tissue sample is obtained from the subject prior to initiation of radiotherapy, chemotherapy or other therapeutic treatment.

“Substantially similar” with respect to nucleic acid or amino acid sequences, means at least about 65% identity between two or more sequences. The term can refer to at least about 70, 75, 80, 85, 90, 91, 92, 93, 94, 95, 96, 97, 98, 99 or greater percent identity. Such identity can be determined using algorithms known in the art, such as the BLAST algorithm.

A biological sample from a patient means a sample from a subject suspected to be affected by a disease. As used herein, the term “subject” refers to any mammal, including both human and other mammals. Preferably, the methods of the present invention are applied to human subjects.

The term “isolated” can refer to a nucleic acid or polypeptide that is substantially free of cellular material, bacterial material, viral material, or culture medium (when produced by recombinant DNA techniques) of their source of origin, or chemical precursors or other chemicals (when chemically synthesized). Moreover, an isolated compound refers to one that can be administered to a subject as an isolated compound; in other words, the compound may not simply be considered “isolated” if it is adhered to a column or embedded in an agarose gel. Moreover, an “isolated nucleic acid fragment” or “isolated peptide” is a nucleic acid or protein fragment that is not naturally occurring as a fragment and/or is not typically in the functional state.

Moieties of the invention, such as polypeptides, peptides, quenchers, or fluorophores may be conjugated or linked covalently or noncovalently to other moieties such as proteins, peptides, supports, fluorescence moieties, or labels. The term “conjugate” is broadly used to define the operative association of one moiety with another agent and is not intended to refer solely to any type of operative association and is particularly not limited to chemical “conjugation.”

The term “providing” is used according to its ordinary meaning “to supply or furnish for use.” In some embodiments, the protein is provided directly by administering the protein, while in other embodiments, the protein is effectively provided by administering a nucleic acid that encodes the protein.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1A-1J. Recombinant RING constructs mimic distinct relative RING arrangements in TRIM5α. (A) Conserved tripartite motif (TRIM) domain arrangement in TRIM5α. (B) NMR structure of the human TRIM5a RING domain monomer (PDB: 2ECV). (C) Cartoon representation of the TRIM5a dimer structure. (D) The monomeric R1 RING construct used in this study. (E) Crystal structure of the rhesus TRIM5a RING (PDB: 4TKP) reveals the four-helix-bundle dimer interface. (F) RING dimerization is thought to occur when multiple TRIM5a dimers come into proximity. (G) RING dimerization is stabilized in the R2 tandem RING construct. (H) Crystal structure of the B-box trimer (PDB: 5IEA) reveals the structural arrangement of three B-box domains at the vertices of the honeycomb-like TRIM5a assemblies (I)(Ganser-Pornillos et al., Proc Natl Acad Sci USA 108, 534-9, 2011; Wagner et al., Elife 5, 2016). (J) Trimeric arrangement of RING domains at the vertices of the TRIM5a honeycomb was mimicked in the R3 construct by fusing RING domain to T4 fibritin foldon, a compact trimerization domain.

FIG. 2A-2C. Conformational repertoire of distinct RING constructs as revealed by NMR spectroscopy. (A)¹⁵N-TROSY HSQC spectra of different RING constructs reveal spectral signatures of RING dimerization. (B) Three representative spectral regions from (A) showing NMR signals that display distinctive chemical shift changes upon transition between the monomeric and the dimeric states of the RING domain. (C) Cartoon representation of the distinct structural conformations observed in different RING constructs.

FIG. 3A-3D. Kinetics of RING-catalyzed ubiquitin discharge as revealed by a FRET ubiquitin discharge assay. (A) A cartoon schematic of the FRET ubiquitin discharge assay used in this study. (B) Kinetics of RING-catalyzed ubiquitin discharge from the heterodimeric UBE2N/V2. (C) Ubiquitin discharge from UBE2W. (D) Ubiquitin discharge was modeled using the Michaelis-Menten equation as described in the text and the initial slopes of the Michaelis-Menten curves were used to quantify E3 activity of different RING constructs.

FIG. 4A-4K. Proximity of three RING domains promotes each of the two distinct steps in the autoubiquitination of TRIM5α: the N-terminal monoubiquitination and the extension of the TRIM5α-anchored K63-linked polyubiquitin chain. (A, B) RING autoubiquitination products visualized by WB of FLAG-tagged RING constructs. (A) Monoubiquitination of RING in the presence of the increasing amounts of UBE2W. (B) RING polyubiquitination in the presence of both UBE2W and the heterodimeric UBE2N/V2. (C) Ubiquitination products of different E2/E3 combinations analyzed side-by-side on the same SDS-PAGE gel. WB was performed with either anti-FLAG antibodies (top panel) or anti-K63-linked-Ub antibodies (bottom panel). (D) N-terminal anchoring of ubiquitin in the R3 WT construct is confirmed by mass spectrometry. (E) N-terminal monoubiquitination is preferred over other modification sites by more than two orders of magnitude. (F) Fluorescent SDS-PAGE analysis of the R3 WT construct C-terminally fluorescently labeled with AF594 reveals that all three RINGs within the trimer are monoubiquitinated by UBE2W. (G) In vitro ubiquitination of R3 WT and purification of the Ub-R3 WT product. (H) Fluorescent SDS-PAGE analysis of the ubiquitination products using AF488-labeled ubiquitin. (I) Ubiquitin discharge from UBE2N/V2 by the R3 WT construct is strongly enhanced by N-terminal monoubiquitination. (J) N-terminal monoubiquitination also alters the products of ubiquitin discharge. (K) Cartoon representation of the two key events in the autoubiquitination of the R3 WT construct: the N-terminal monoubiquitination of the RING trimer by UBE2W and the subsequent extension of RING-anchored K63-linked polyubiquitin chain by UBE2N/V2.

FIG. 5. Molecular mechanism that promotes TRIM5a autoubiquitination upon binding to the retroviral capsid. Cooperativity between SPRY:capsid interactions and B-box:B-box interactions forms the basis of the pattern recognition functionality and promotes assembly of TRIM5a on the capsid surface¹⁰. Proximity of three RING domains at the vertices of the TRIM5a honeycomb enhances the rates of the UBE2W-mediated and UBE2N/V1-mediated autoubiquitination events. Relative arrangement of protein subunits in the complexes mediating these events can be modeled with high confidence based on extensive existing structural data. The models (insets in the upper right and lower right corners of the figure) illustrate how proximity of the third RING and the flexibility of its backbone facilitate ubiquitin transfer in the two complexes.

FIG. 6A-6B. An illustration of example mechanisms for (A) FRET assays and (B) quenched fluorescence assays.

FIG. 7A-7C. An illustration of (A) the molecular mechanism of one example of a FRET assay, (B) results of an assay, and (C) analysis of the results of the assay.

FIG. 8. An illustration of one example of a protein labeling mechanism using sortase activity.

FIG. 9A-9D. An illustration of one example of a FRET assay of E1 activity. (A) A cartoon depicting the assay in which a FRET-active E2-Ubl conjugate is formed when a fluorescently labeled E2 protein and a fluorescently labeled Ubl protein are incubated in the presence of a E1 enzyme. In this particular example 1 μM AF488-labeled Ub (human ubiquitin) and 1 μM AF594-labeled E2 (human UBE2N) were incubated in the presence of 5 mM ATP and increasing concentrations (0 μM, 0.06 μM, 0.13 μM, 0.25 μM) of E1 (human UBA1). (B) Measurement of fluorescence intensity using 480/20 nm excitation and 630/20 nm emission settings reveals an increase in the FRET signal vs time as the FRET-active E2-Ub conjugate is being formed in the reaction. (C,D) SDS-PAGE analysis of the reaction products formed in the reaction as a function of the reaction time. The SDS-PAGE gel imaged using AF488 fluorescence (C) and AF594 fluorescence (D) reveals that the E2-Ub conjugate product is formed from the two substrates of the reaction, the AF488-labeled ubiquitin and AF594-labeled UBE2N.

FIG. 10A-10C. An illustration of one example of a quenched fluorescent assay of E1 activity. (A) A cartoon depicting the assay in which a quenched FRET-active E2-Ubl conjugate is formed when an E2 protein labeled with a fluorescence quencher (SY9) and a fluorescently labeled Ubl protein are incubated in the presence of a E1 enzyme and ATP. In this particular example 1 μM AF488-labeled Ub (human ubiquitin) and 1 μM SY9-labeled E2 (human UBE2N) were incubated in the presence of 5 mM ATP and increasing concentrations (0 μM, 0.06 μM, 0.13 μM, 0.25 μM) of E1 (human UBA1). (B) Measurement of fluorescence intensity using 480/20 nm excitation and 520/20 nm emission settings reveals a decrease in the intensity of the AF488 fluorescence signal vs time as the quenched FRET-active E2-Ub conjugate is being formed in the reaction. (C) SDS-PAGE analysis by AF488 fluorescence reveals that the E2-Ub conjugate product is formed from the two substrates of the reaction, the AF488-labeled ubiquitin and SY9-labeled UBE2N.

FIG. 11A-11D. An illustration of one example of a FRET assay of E3 activity. (A) A cartoon depicting the assay in which a FRET-active E2-Ubl conjugate is dissociated upon incubation with a substrate protein and an E3 ubiquitin ligase. In this example 0.1 μM of the FRET-active E2-Ubl conjugate formed between AF488-labeled Ub (human ubiquitin) and AF594-labeled E2 (human UBE2N) was incubated with 0.1 μM of human UBE2V2(MMS2), 5 μM substrate protein (unlabeled human ubiquitin) and increasing concentrations (0 μM, 2.5 μM, 5.0 μM, 10 μM, 20 μM) of E3 ligase (human TRAF6 RING domain). (B) Measurement of fluorescence intensity using 480/20 nm excitation and 630/20 nm emission settings reveals a decrease in the intensity of the FRET signal vs time as the FRET-active E2-Ub conjugate is being dissociated in the reaction. (C,D) SDS-PAGE analysis of the reaction products formed in the reaction as a function of the reaction time. The SDS-PAGE gel imaged using AF488 fluorescence (C) and AF594 fluorescence (D) reveals that the E2-Ub conjugate is dissociated in the reaction, in which the AF488-labeled ubiquitin is incorporated into the di-ubiquitin product and the AF594-labeled UBE2N is released in the free form.

FIG. 12A-12C. An illustration of one example of a quenched fluorescent assay of E3 activity. (A) A cartoon depicting the assay in which a FRET-active E2-Ubl conjugate is dissociated upon incubation with a substrate protein and an E3 ubiquitin ligase. In this example 0.1 μM of the FRET-active E2-Ubl conjugate formed between AF488-labeled Ub (human ubiquitin) and SY9-labeled E2 (human UBE2N) was incubated with 0.1 μM of human UBE2V2(MMS2), 5 μM substrate protein (unlabeled human ubiquitin) and increasing concentrations (0 μM, 2.5 μM, 5.0 μM, 10 μM, 20 μM) of E3 ligase (human TRAF6 RING domain). (B) Measurement of fluorescence intensity using 480/20 nm excitation and 520/20 nm emission settings reveals an increase in the intensity of the AF488 fluorescence signal vs time as the quenched FRET-active E2-Ub conjugate is being dissociated in the reaction. (C) SDS-PAGE analysis by AF488 fluorescence reveals that the E2-Ub conjugate is dissociated in the reaction and the AF488-labeled ubiquitin is transferred onto the unlabeled ubiquitin substrate protein forming the di-ubiquitin product.

FIG. 13A-13D. Kinetics and products of ubiquitin transfer reactions catalyzed by TRIM5 RING constructs. (A) Kinetics and products of ubiquitin discharge from the AF594-UBE2W˜AF488-Ub conjugate (0.1 μM) catalyzed by R2-WT ([RING]=1 μM; [dimer]=0.5 μM) or R3-WT constructs ([RING]=0.5 μM; [trimer]=0.17 μM) in the presence of [N5-Ub]=5 μM. (B) Cartoon representation of the UBE2W reactions and the corresponding products. (C) Kinetics and products of ubiquitin discharge from the AF594-UBE2N˜AF488-Ub/UBE2V2 conjugate (0.1 μM) catalyzed by R3-WT ([RING]=0.5 μM; [trimer]=0.17 μM) or Ub-R3-WT constructs ([RING]=0.5 μM; [trimer]=0.17 μM) in the presence of [N5-Ub]=5 μM. (D) Cartoon representation of the UBE2N/V2 reactions and the corresponding products.

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be an example of that embodiment, and not intended to imply that the scope of the disclosure, including the claims, is limited to that embodiment.

### I. PROTEIN UBIQUITINATION

Post-translational, covalent conjugation of ubiquitin or ubiquitin-like proteins is a major eukaryotic regulatory mechanism. Conjugation of Ub/Ubl requires an enzymatic cascade involving an E1 activating enzyme (e.g., U13A1 (Genbank accession No. X55386), E2 conjugating enzymes, and E3 ligases. Several steps are involved in ubiquitin (Ub) conjugation and protein degradation. First, Ub/Ubl is activated by a ubiquitin-activating enzyme (E1) in an ATP dependent manner. Activation involves binding of the C-terminus of Ub/Ubl to the thiol group of a cysteine residue of E1. Activated Ub/Ubl is subsequently transferred to one of several Ub-conjugating enzymes (E2). Each E2 has a recognition subunit which allows it to interact with proteins carrying a particular degradation signal. E2 links the Ub/Ubl molecule through its C-terminal glycine to an internal lysine of the target protein. Different ubiquitin-dependent proteolytic pathways employ structurally similar, but distinct, E2s, and in some instances, accessory factors known as ubiquitin-ligases or E3s, are required to work in conjunction with E2s for recognition of certain substrates.

Prior to activation, Ub/Ubl is usually expressed as a protein precursor composed of an N-terminal ubiquitin and a C-terminal extension protein (CEP) or as a polyubiquitin protein with Ub/Ubl monomers attached head to tail. CEPs have characteristics of a variety of regulatory proteins; most are highly basic, contain up to 30% lysine and arginine residues, and have nucleic acid-binding domains. The fusion protein is an important intermediate which appears to mediate co-regulation of the cell's translational and protein degradation activities, as well as localization of the inactive enzyme to specific cellular sites. Once delivered, C-terminal hydrolases cleave the fusion protein to release a functional Ub/Ubl.

The E2s are important for substrate specificity in several ubiquitin conjugating pathways. In addition, E2s are important for creating specific linkages between ubiquitin monomers in a polyubiquitin chain. For example, UBE2W is thought to function as a specialized E2 that has specificity for attaching ubiquitin to the N-terminus of the substrate protein. Another example of a specialized E2 is UBE2N, which normally forms heterodimers with UBE2V1 or UBE2V2 and in these heterodimeric complexes is specific for attaching ubiquitin to the lysine 63 (K63) residue of another ubiquitin and thus mediates synthesis of K63-linked polyubiquitin chains. All E2s have a conserved ubiquitin conjugation (UBC) domain of approximately 16 kD, at least 35% identity with each other, and contain a centrally located cysteine residue which is necessary for the formation of the covalent thioester bond that links the E2 and Ubl in the E2-Ubl conjugate.

### A. Ubiquitin

Ubiquitin (Ub) is an abundant 76 amino acid residue polypeptide that is found in all eukaryotic cells. The ubiquitin polypeptide is characterized by a carboxy-terminal glycine residue that is activated by ATP to a high-energy thiol-ester intermediate in a reaction catalyzed by a ubiquitin-activating enzyme (E1). The term “ubiquitin” includes within its scope all known ubiquitin homologs of vertebrate or invertebrate origin. Examples of ubiquitin polypeptides as referred to herein include the human ubiquitin polypeptide (e.g., MQIFVKTLTGKTITLEVEPSDTIENVKAKIQDKEGIPPDQQRLIFAGKQLEDGRTLSDYNI QKESTLHLVLRLRGGMQIFVKTLTGKTITLEVEPSDTIENVKAKIQDKEGIPPDQQRLIFA GKQLEDGRTLSDYNIQKESTLHLVLRLRGGMQIFVKTLTGKTITLEVEPSDTIENVKAKI QDKEGIPPDQQRLIFAGKQLEDGRTLSDYNIQKESTLHLVLRLRGGC, SEQ ID NO:2) which is encoded by the human ubiquitin encoding nucleic acid sequence (GenBank Accession Numbers: U49869, X04803) as well as all equivalents. Equivalent ubiquitin polypeptides are understood to include those sequences that differ by one or more amino acid substitutions, additions or deletions, such as allelic variants.

The term “ubiquitin-like protein (Ubl)” refers to a group of naturally occurring proteins, not otherwise describable as ubiquitin equivalents, but which nonetheless show strong amino acid homology to ubiquitin. As used herein this term includes the polypeptides SUMO1-4, NEDD8, ATG8, ATG12, URM1, UFM1, FAT10, ISG15 and the like. Similar to ubiquitin, these “ubiquitin-like proteins” are covalently attached to substrate proteins as a form of posttranslational modification, whose mechanism parallels that of ubiquitin attachment described above. Ubiquitin-related proteins include, but are not limited to SUMO1 (human variant Accession No. P63165.1), SUMO2 (human variant Accession No. P61956.3), SUMO3 (human variant Accession No. P55854.2), SUMO4 (human variant Accession No. Q6EEV6.2), NEDD8 (human variant Accession No. Q15843.1), ATG8 (human variant Accession No. Q1E4K5.1), ATG12 (human variant Accession No. 094817.1), URM1 (human variant Accession No. Q9BTM9.1), UFM1 (human variant Accession No. P61960.1), FAT10 (human variant Accession No. 015205.2) and ISG15 (human variant Accession No. P05161.5).

### B. Ubiquitin Activating Enzyme (E1)

Ubiquitin-activating enzymes (E1 enzymes) are a family of related proteins that activate ubiquitin or another ubiquitin-like protein by catalyzing synthesis of a Ub/Ubl intermediate containing a high-energy bond. E1 proteins useful in the invention include, but are not limited to, mammalian E1 (e.g., human, mouse and rat E1, etc.) or Els of other species such as plant (e.g., wheat), zebrafish and C. elegans. In a preferred embodiment, E1 is a human E1 having the amino acid sequence recited in SEQ ID NO:3 (CAA40296.1-MSSSPLSKKRRVSGPDPKPGSNCSPAQSVLSEVPSVPTNGMAKNGSEADIDEGLYSRQL YVLGHEAMKRLQTSSVLVSGLRGLGVEIAKNIILGGVKAVTLHDQGTAQWADLSSQFY LREEDIGKNRAEVSQPRLAELNSYVPVTAYTGPLVEDFLSGFQVVVLTNTPLEDQLRVG EFCHNRGIKLVVAGTRGLFGQLFCDFGEEMILTDSNGEQPLSAMVSMVTKDNPGVVTC LDEARHGFESGDFVSFSEVQGMVELNGNQPMEIKVLGPYTFSICDTSNFSDYIRGGIVSQ VKVPKKISFKSLVASLAEPDFVVTDFAKFSRPAQLHIGFQALHQFCAQHGRPPRPRNEED AAELVALAQAVNARALPAVQQNNLDEDLIRKLAYVAAGDLAPINAFIGGLAAQEVMK ACSGKFMPIMQWLYFDALECLPQDKEVLTEDKCLQRQNRYDGQVAVFGSDLQEKLGK QKYFLVGAGAIGCELLKNFAMIGLGCGEGGEIIVTDMDTIEKSNLNRQFLFRPWDVTKL KSDTAAAAVRQMNPHIRVTSHQNRVGPDTERIYDDDFFQNLDGVANALDNVDARMYM DRRCVYYRKPLLESGTLGTKGNVQVVIPFLTESYSSSQDPPEKSIPICTLKNFPNAIEHTLQ WARDEFEGLFKQPAENVNQYLTDPKFVERTLRLAGTQPLEVLEAVQRSLVLQRPQTWA DCVTWACHHWHTQYSNNIRQLLHNFPPDQLTSSGAPFWSGPKRCPHPLTFDVNNPLHL DYVMAAANLFAQTYGLTGSQDRAAVATFLQSVQVPEFTPKSGVKIHVSDQELQSANAS VDDSRLEELKATLPSPDKLPGFKMYPIDFEKDDDSNFHMDFIVAASNLRAENYDIPSADR HKSKLIAGKIIPAIATTTAAVVGLVCLELYKVVQGHRQLDSYKNGFLNLALPFFGFSEPL AAPRHQYYNQEWTLWDRFEVQGLQPNGEEMTLKQFLDYFKTEHKLEITMLSQGVSML YSFFMPAAKLKERLDQPMTEIVSRVSKRKLGRHVRALVLELCCNDESGEDVEVPYVRY TIR). Els are commercially available from different suppliers, e.g., Affiniti Research Products (Exeter, U.K.), R&D Systems Inc (Minneapolis, Minnesota, USA) and others. Variants of the cited E1 proteins are also included in the term “E1”.

### C. Ubiquitin Conjugating Enzyme (E2)

E2 proteins useful in the invention include, but are not limited to, mammalian E2 (e.g., human, mouse and rat E2s, etc.) or E2s of other species such as plant (e.g., wheat), zebrafish and C. elegans. The skilled artisan will appreciate that many different E2 proteins and isozymes are known and may be used in the present invention, provided that the E2 forms a covalent E2-Ub/Ubl conjugate with ubiquitin or another ubiquitin-like protein. Also specifically included within the term “E2” are variants of E2. In one example the E2 protein is the human E2 protein UBE2D1 that has the amino acid sequence P51668.1-MALKRIQKELSDLQRDPPAHCSAGPVGDDLFHWQATIMGPPDSAYQGGVFFLTVHFPT DYPFKPPKIAFTTKIYHPNINSNGSICLDILRSQWSPALTVSKVLLSICSLLCDPNPDDPLVP DIAQIYKSDKEKYNRHAREWTQKYAM (SEQ ID NO.4). In another example the E2 protein is the human E2 protein UBE2N (Accession No. P61088.1). In another example the E3 protein is the human E2 protein UBE2W (Accession No. P61088.1). While some E2s are E3-specific, i.e., they work with only certain E3s, other E2s are known to work with many different E3s. In another embodiment, the E2 of the present invention contains a panel of E2 proteins. In a preferred embodiment, the panel of E2s contains 2-20 E2 proteins. In a more preferred embodiment, the panel of E2s contains 3-10 E2 proteins. The inclusion of multiple E2s in an assay system would allow the detection of most, if not all, E3 activities.

### D. Ubiquitin Ligase (E3)

In certain aspects the E3 is an unknown E3 in a sample. In other aspects a known E3 or a panel of known E3s are utilized. By “E3” is meant an E3 ubiquitin ligase comprising one or more components that possesses the ability to catalyze transfer of ubiquitin or another ubiquitin-like protein from a E2-Ub/Ubl conjugate onto a substrate protein, where the substrate protein can be the E3 itself, another ubiquitin protein or any other cellular protein. Suitable E3s include, but are not limited to, RING finger-, Homologous to the E6-AP Carboxyl Terminus (HECT)-, U box-, PHD finger-type of E3s. In certain aspects E3 is the human E3, TRIM5alpha, having an amino acid sequence of NP_149023.2-MASGILVNVKEEVTCPICLELLTQPLSLDCGHS FCQACLTANHKKSMLDKGESSCPVCRISYQPENIRPNRHVANIVEKLREVKLSPEGQKV DHCARHGEKLLLFCQEDGKVICWLCERSQEHRGHHTFLTEEVAREYQVKLQAALEMLR QKQQEAEELEADIREEKASWKTQIQYDKTNVLADFEQLRDILDWEESNELQNLEKEEED ILKSLTNSETEMVQQTQSLRELISDLEHRLQGSVMELLQGVDGVIKRTENVTLKKPETFP KNQRRVFRAPDLKGMLEVFRELTDVRRYWVDVTVAPNNISCAVISEDKRQVSSPKPQII YGARGTRYQTFVNFNYCTGILGSQSITSGKHYWEVDVSKKTAWILGVCAGFQPDAMCN IEKNENYQPKYGYWVIGLEEGVKCSAFQDSSFHTPSVPFIVPLSVIICPDRVGVFLDYEAC TVSFFNITNHGFLIYKFSHCSFSQPVFPYLNPRKCGVPMTLCSPSS (SEQ ID NO:5)). In another example the E3 protein is the TRIM5alpha of the rhesus macaque (Accession No. Q0PF16.2).

### E. Recombinant Proteins

One or more of ubiquitin or ubiquitin-like protein, E1, E2, and/or E3 can be isolated and/or recombinant proteins. A “recombinant protein” is a protein made using recombinant techniques, i.e., through the expression of a recombinant nucleic acid. A recombinant protein is distinguished from naturally occurring protein by at least one or more characteristics. For example, the protein may be isolated or purified away from some or all of the proteins and compounds with which it is normally associated in its wild-type host. A substantially pure protein comprises at least about 75%, 80%, or 90% by weight of the protein. A recombinant protein includes the production of a protein from one organism in a different organism or host cell. Alternatively, the protein may be made at a significantly higher concentration than is normally seen using of an inducible promoter or high expression promoter, such that the protein is made at increased concentration levels. Alternatively, the protein may be in a form not normally found in nature, as in the addition of an epitope tag or amino acid substitutions, insertions and deletions.

### F. Protein Variants

Compositions, methods, and kits can contain protein variants, for example ubiquitin, E1, E2, and/or E3 variants. These variants fall into one or more of three classes: substitutional, insertional or deletional variants. These variants ordinarily are prepared by site specific mutagenesis of nucleotides in the DNA encoding a protein of the present compositions, using cassette or polymerase chain reaction (PCR) mutagenesis or other techniques well known in the art, to produce DNA encoding the variant, and thereafter expressing the DNA in recombinant cell culture. Some variants may exhibit the same qualitative biological activity as the naturally occurring analog. Other variants may be deficient in one or more functionalities of the wildtype protein and as such can be used as negative controls in experiments that depend on such functionality.

Amino acid substitutions are typically of single residues; insertions usually will be on the order of from about 1 to 20 amino acids, although considerably larger insertions may be tolerated. Deletions range from about 1 to about 20 residues, although in some cases deletions may be much larger.

Substitutions, deletions, insertions or any combination thereof may be used to arrive at a final derivative or variant. Generally, these changes are done on a few amino acids to minimize the alteration of the molecule. However, larger changes may be tolerated in certain circumstances. When small alterations in the characteristics of the protein are desired, substitutions are generally made in accordance with the following substitutions: Ala to Ser, Arg to Lys, Asn to Gln or His, Asp to Glu, Cys to Ser or Ala, Gln to Asn, Glu to Asp, Gly to Pro, His to Asn or Gln, Ile to Leu or Val, Leu to Ile or Val, Lys to Arg or Gln or Glu, Met to Leu or Ile, Phe to Met or Leu or Tyr, Ser to Thr, Thr to Ser, Trp to Tyr, Tyr to Trp or Phe, and/or Val to Ile or Leu.

### II. FRET-ACTIVE E2-UB/UBL CONJUGATES

E2 ubiquitin conjugating enzyme is one of the components involved in modification of proteins with Ub/Ubl (ubiquitin or ubiquitin-like protein). E2s form covalent conjugates with Ub/Ubl, in which the carboxyl terminus of the Ub/Ubl is linked to the active site cysteine of E2 via a thioester bond. An example of an E2 ubiquitin conjugating enzyme includes those encoded by the following genes: UBE2A, UBE2B, UBE2C, UBE2D1, UBE2D2, UBE2D3, UBE2E1, UBE2E2, UBE2E3, UBE2F, UBE2G1, UBE2G2, UBE2H, UBE2I, UBE2J1, UBE2J2, UBE2K, UBE2L3, UBE2L6, UBE2M, UBE2N, UBE20, UBE2Q1, UBE2Q2, UBE2R1, UBE2R2, UBE2S, UBE2T, UBE2U, UBE2V1, UBE2V2, UBE2Z, ATG2, BIRC5, and UFC, and homologs or portions thereof.

The term “ubiquitin” or “Ub” refers to ubiquitin in accordance with the amino acid sequence (MQIFVKTLTGKTITLEVEPSDTIENVKAKIQDKEGIPPDQQRLIFAGKQLE DGRTLSDYNIQKESTLHLVLRLRGG (SEQ ID NO:1) or to proteins or proteins having a domain or segment with at least 40, 50, 60, 70, 80, 90, 95, to 100% amino acid identity to SEQ ID NO: 1. Particularly preferred are ubiquitin molecules from mammals, e.g., humans, primates, pigs, and rodents. On the other hand, the ubiquitin origin is not relevant since according to the art all eukaryotic ubiquitins are highly conserved and the mammalian ubiquitins examined up to now are even identical with respect to their amino acid sequence. In addition, ubiquitin from any other eukaryotic source can be used. For instance ubiquitin of yeast differs only in three amino acids from the wild-type human ubiquitin (SEQ ID NO: 1).

The term “ubiquitin-like protein” refers to a group of naturally occurring proteins, not otherwise describable as ubiquitin equivalents, but which nonetheless show strong amino acid homology to ubiquitin. As used herein this term includes the polypeptides SUMO1-4, NEDD8, ATG8, ATG12, URM1, UFM1, FAT10, ISG15 and the like. Similar to ubiquitin, these “ubiquitin-like proteins” are covalently attached to substrate proteins as a form of posttranslational modification, whose mechanism parallels that of ubiquitin attachment described above. Ubiquitin-related proteins include, but are not limited to SUMO1 (human variant Accession No. P63165.1), SUMO2 (human variant Accession No. P61956.3), SUMO3 (human variant Accession No. P55854.2), SUMO4 (human variant Accession No. Q6EEV6.2), NEDD8 (human variant Accession No. Q15843.1), ATG8 (human variant Accession No. Q1E4K5.1), ATG12 (human variant Accession No. 094817.1), URM1 (human variant Accession No. Q9BTM9.1), UFM1 (human variant Accession No. P61960.1), FAT10 (human variant Accession No. 015205.2) and ISG15 (human variant Accession No. P05161.5).

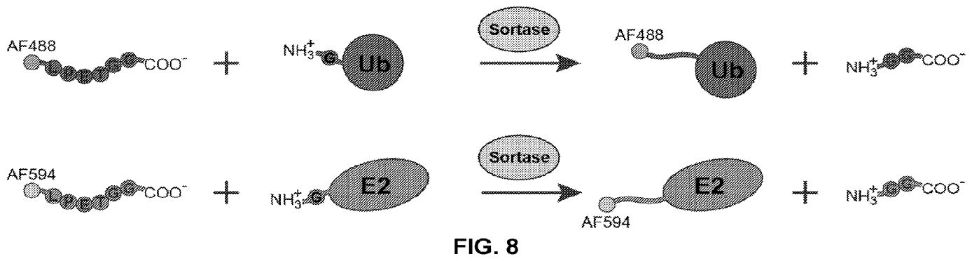

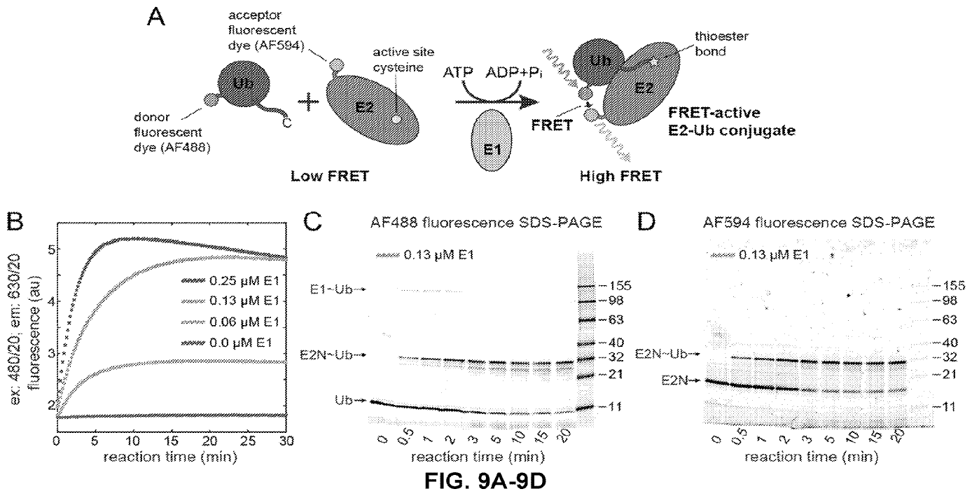

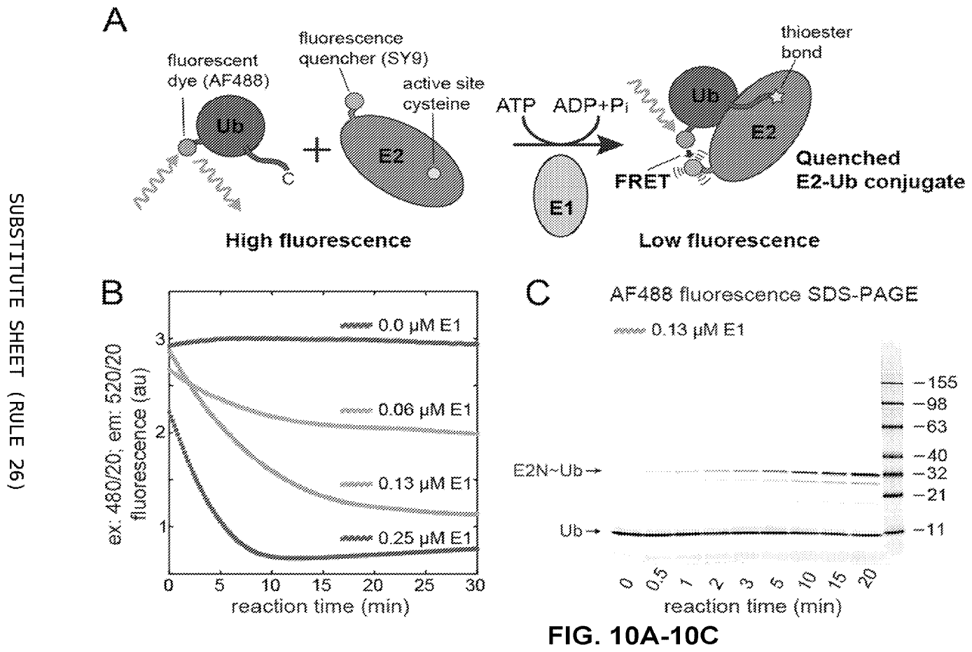

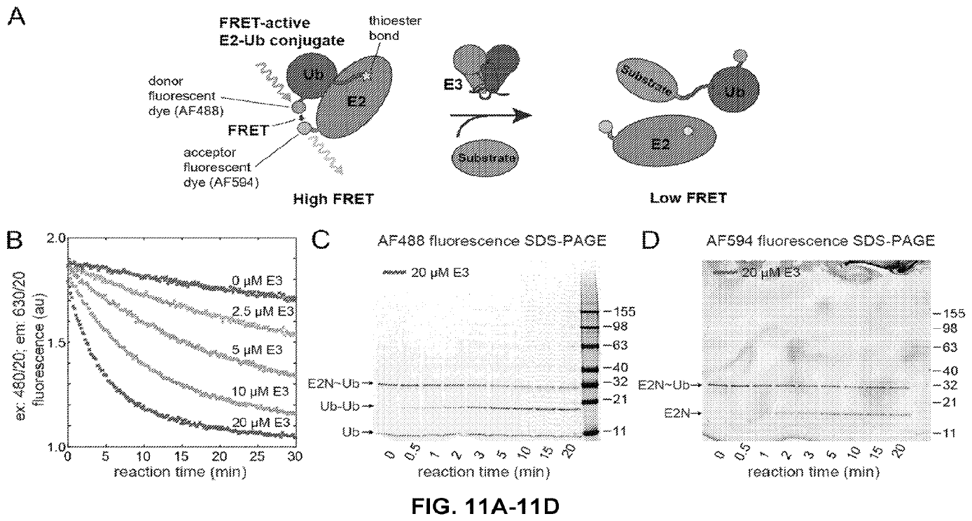

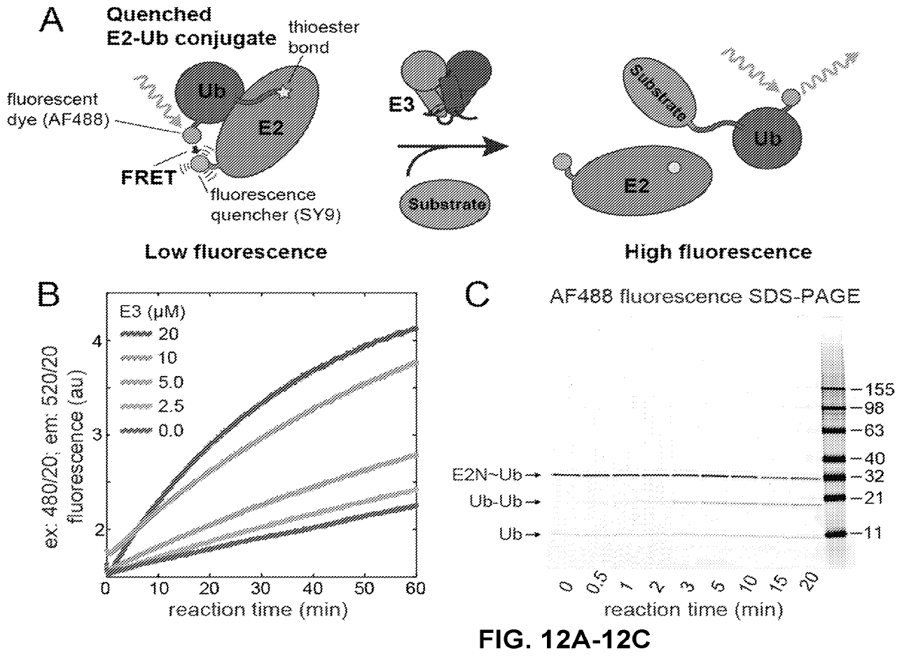

Fluorescently labeled Ubl. Labeling a ubiquitin protein or ubiquitin-like protein with a first fluorophore can be done using any of several existing methods for site-specific, high-efficiency fluorescent labeling of proteins. High-efficiency fluorescent labeling is referring to a method that allows for more than 50% of the total protein in the sample to be covalently linked to a fluorophore. Site-specific fluorescent labeling is referring to a method that allows attachment of a fluorophore at one or more specific desired locations. In one example, fluorescently labeled Ubl can be prepared using a sortase-catalyzed transpeptidation (Theile et al., Nat Protoc, 8(9):1800-7, 2013) as follows: One reactant is a recombinant or isolated Ub, in certain aspects the Ub will have an engineered N-terminal extension in which glycine is the N-terminal residue. A second reactant is a synthetic short peptide (for example KLPETGG (SEQ ID NO:6)) that contains a sortase recognition sequence (e.g., LPXTG where X is any amino acid) is covalently modified with a first fluorophore (for example an Alexa Fluor 488 NHS ester is reacted with the N-terminal lysine residue of the peptide). Other methods for this labeling reaction are known in the art. The Ub and synthetic peptide are mixed in the presence of a sortase enzyme. The sortase catalyzed transpeptidation reaction yields Ubiquitin covalently modified with the first fluorophore (FIG. 8).

Other methods for site-specific, high-efficiency labeling of proteins with fluorophores are known in the art. Some of the methods utilize enzymes other than sortase for site specific attachment of fluorophores to proteins. Transglutaminase-catalyzed site-specific conjugation of proteins with fluorophores is one example (Lin and Ting, J Am Chem Soc. 128(14):4542-3, 2006). In another example, phosphopantetheinyl transferases are used to conjugate fluorophores to short peptide tags incorporated into the amino acid sequence of a protein (Zhou et al., ACS Chem Biol, 2(5):337-46, 2007). In another example, biotin ligases have been used for labeling of proteins with fluorophores (Chen et al., Nat Methods, 2(2):99-104, 2005). Other methods utilize incorporation of a natural or unnatural amino acid with unique reactivity at a specific location in a protein and subsequently forming a covalent bond between this amino acid and a fluorophore containing a chemical group that specifically reacts with this amino acid. One example is introducing a cysteine residue into a protein that is normally devoid of cysteine residues on its surface, or into a protein in which all other surface-exposed cysteines have been mutated. A fluorophore containing a sulfhydryl-reactive group (e.g., malemide) is then reacted with the protein and forms a covalent bond with the cysteine residue. In another example, an unnatural amino acid with unique reactivity (e.g., containing an azide group or a strained alkyne group) is introduced into protein at a desired location using genetic code expansion technology (Plass et al., Angew Chem Int Ed Engl, 50(17):3878-81, 2011; Nikic et al. Angew Chem Int Ed Engl, 53(8):2245-9, 2014). A fluorophore containing an appropriate reactive group can subsequently be reacted with this unnatural amino acid to form a covalent bond with the protein (e.g., using click chemistry or another biorthogonal chemical reaction).

Preparation of E2 covalently modified with a second fluorophore. E2 is labeled with a second fluorophore (e.g., a fluorescent acceptor dye Alexa Fluor 594) using, for example, a sortase-catalyzed transpeptidation as follows: One reactant is a E2 protein (e.g., UBE2N, UBE2D and UBE2W) having an engineered N-terminal extension in which glycine is the N-terminal residue. A second reactant is a synthetic short peptide (e.g., KLPETGG (SEQ ID NO:6)) containing a sortase recognition sequence (e.g., LPXTG where X is any amino acid (SEQ ID NO:7)) is covalently modified with the second fluorophore (e.g., Alexa Fluor 594 NHS ester is reacted with the N-terminal lysine residue of the peptide. Other methods for this labeling reaction are known in the art. The E2 protein and synthetic peptide are mixed in the presence of a sortase enzyme. Sortase catalyzed transpeptidation reaction yields E2 covalently modified with a second fluorophore (FIG. 8).

Fluorescently labeled E2-Ub conjugates. Ubiquitin covalently modified with a first fluorophore (described above) is mixed with E2 covalently modified with a second fluorophore (described above) in the presence of a E1 ubiquitin activating enzyme and ATP producing a FRET-active E2-Ub/Ubl conjugate, in which the first fluorophore comes in proximity to the second fluorophore. Other embodiments can use other known methods for covalent modification of proteins with fluorescent dyes.

Labeled E2-Ub conjugates for ubiquitin discharge assays can be prepared by incubating E1, donor or first fluorophore or quencher-labeled E2, acceptor or second fluorophore or quencher-labeled Ub, and ATP in a conjugation buffer under conditions that form a covalent bond between E2 and Ub. A conjugation reaction can be incubated for 5, 10, 15, 20, 25, 30, 35, 40, 45, 50, 55, 60 min or more including all times and ranges there between. The conjugation reaction can be performed at temperature of 25, 30, 35, 37, 40, 45° C., including all temperatures and ranges there between. The reactions can be terminated by depleting ATP with apyrase. The terminated reactions can be diluted and dispensed into an appropriate assay format, for example a 384-well fluorescence microplate.

### III. FRET ASSAYS AND QUENCHED FLUORESCENCE ASSAYS UTILIZING FRET-ACTIVE E2-UB/UBL CONJUGATES AS SUBSTRATES, PRODUCTS OR INTERMEDIATES

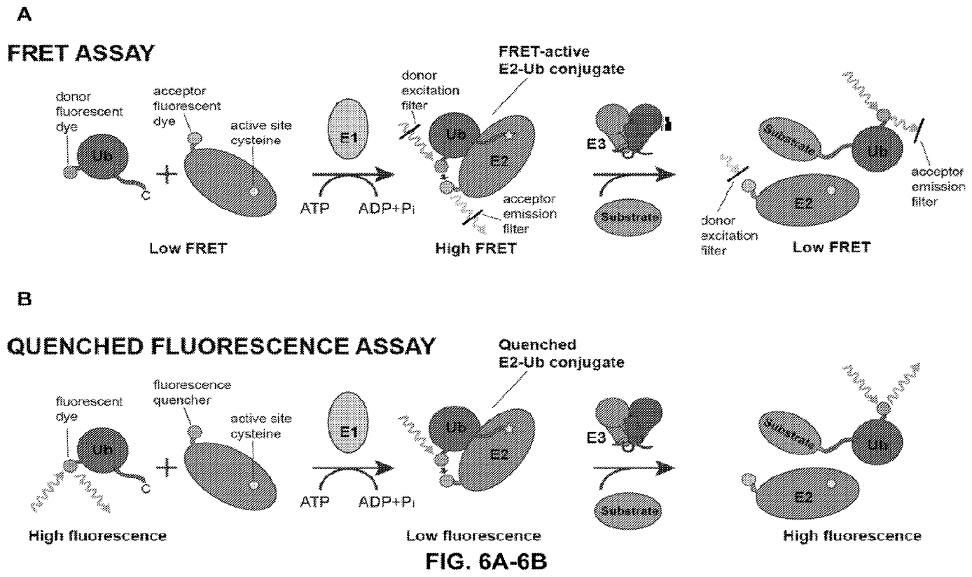

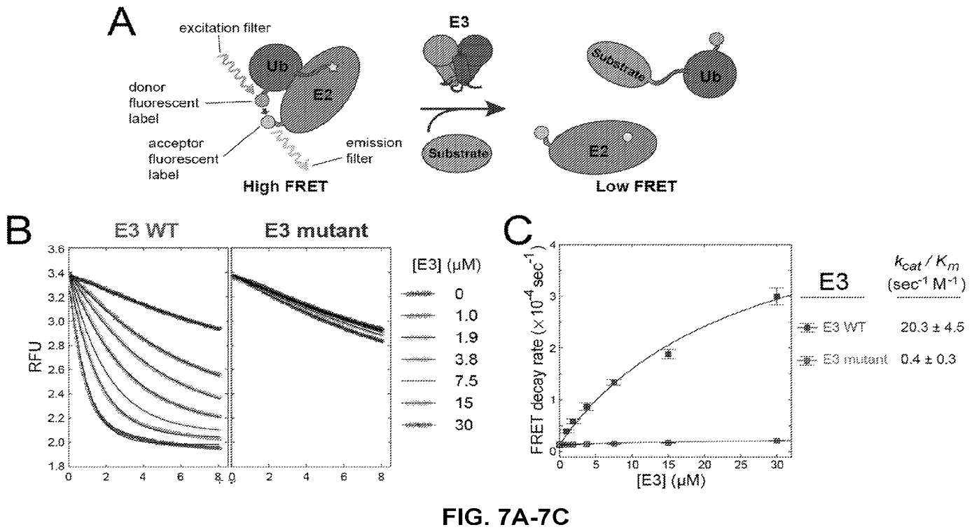

FRET Assay for measuring E3 ubiquitin ligase activity. This assay improves on a traditional ubiquitin discharge assay, by replacing the SDS-PAGE readout with a real-time fluorescence readout. FRET-active E2-Ub/Ubl conjugates (e.g., AF488-Ub-AF594-UBE2D, AF488-Ub-AF594-UBE2N and AF488-Ub-AF594-UBE2W) prepared as described above can be mixed with different recombinant constructs of an E3 ligase (e.g., TRIM5alpha E3 ligase, a protein that potently protects rhesus monkeys against HIV infection) and an appropriate substrate (e.g., in the case of TRIM5alpha, the ligase undergoes auto-ubiquitination, so it acts as its own substrate). The signal (e.g., FRET signal) of a FRET-active E2-Ub/Ubl conjugate can be monitored by illuminating the sample with light of particular wavelength (e.g., using a 480 nm/20 nm bandpass filter as an excitation filter) that matches the excitation wavelength of the donor fluorophore and quantifying the emitted light of an appropriate wavelength (e.g., using a 645 nm/40 nm bandpass filter (emission filter)) that matches the wavelength of the light emitted by the acceptor fluorophore. During the reaction catalyzed by the E3 ligase the FRET-active E2-Ubl conjugate dissociates when the covalent bond between E2 and Ubl is broken and a new bond between Ubl and the substrate is formed. Consequently, the intensity of the observed emitted fluorescence signal decays over time (e.g., see FIG. 6 for examples), and the decay in the signal can be used to quantify the enzymatic activity of the E3 ligase (see FIG. 7C for an example).

The E3 activity FRET assay can also be performed in the time resolved format (TR-FRET). For this purpose, the FRET-active E2-Ub/Ubl conjugate is prepared using a pair of fluorophores commonly used for time resolved FRET measurements (e.g., Europium cryptate as the donor fluorophore and an appropriately matched acceptor fluorophore, e.g., Alexa Fluor 647). The assay is performed the same way as described above, but the FRET signal is detected using a time-resolved fluorimeter. TR-FRET assays may improve the signal to noise of FRET detection because they minimize the background signal arising from direct excitation of the acceptor fluorophore by the excitation light.

Quenched fluorescence assay for E3 ligase activity. Another example of a FRET-active E2-Ubl conjugate is the conjugate in which one of the fluorophores is a fluorescence quencher This type of the FRET-active reagent is often referred to as a “quenched-fluorescence substrate” or “internally quenched fluorescent substrate”. In one example, the FRET-active E2-Ub/Ubl conjugate is made from ubiquitin labeled with AF488 and E2 labeled with a SY9 dark quencher (AF488-Ub-SY9-UBE2D, AF488-Ub-SY9-UBE2N and AF488-Ub-SY9-UBE2W) (SY9 is a generic version of the QSY-9 quencher originally developed by Molecular Probes (ThermoFisher product Q20131)). These conjugates can be mixed with one or more E3 ligase (e.g., different recombinant constructs of the TRIM5alpha E3 ligase of the rhesus monkey) and an appropriate substrate (e.g., in the case of TRIM5alpha, the ligase undergoes auto-ubiquitination, so it acts as its own substrate). The AF488 fluorescence signal is observed using a standard set of the excitation/emission filters used for measuring fluorescence of GFP or fluoresceine. The observed increase in fluorescence intensity results from the E3-catalyzed dissociation of the FRET-active (internally quenched fluorescent) E2-Ubl conjugate. Once the E2-Ubl conjugate is dissociated the AF488 fluorescence is no longer quenched and a strong increase in fluorescence intensity is observed.

FRET assay for E1 ubiquitin activating enzyme activity. Ubiquitin covalently modified with a first fluorophore (e.g., 488 as described) can be mixed with E2 covalently modified with a second fluorophore (e.g., AF594 as described above) in the presence of an E1 ubiquitin activating enzyme and ATP. The FRET signal of the FRET-active E2-Ubl conjugate formed in this reaction is monitored by illuminating the sample with light of a particular wavelength using a 480 nm/20 nm bandpass filter (excitation filter) and quantifying the emitted light of particular wavelength using a 645 nm/40 nm bandpass filter (emission filter). The increase of the FRET signal observed over time results from the E1-catalyzed formation of the FRET-active E2-Ubl conjugate and can be used to quantify the enzymatic activity of the E1.

The E1 FRET assay can also be performed in the time resolved format (TR-FRET) as described above.

Quenched fluorescence assay for E1 ubiquitin activating enzyme activity. Ubiquitin covalently modified with a first fluorophore, fluorophore of a quencher pair (e.g., AF488 as described above) can be mixed with E2 covalently modified with second fluorophore, quencher or fluorophore (e.g., SY9 quencher as described above) in the presence of a E1 ubiquitin activating enzyme and ATP. The fluorescence signal is observed using a standard set of the excitation/emission filters. An observed decrease in fluorescence intensity results from the E1-catalyzed formation of the FRET-active (internally quenched fluorescent) E2-Ubl conjugate and can be used to quantify the enzymatic activity of the E1.

E3 Ubiquitin Ligase. “E3 ubiquitin ligase” (also known as “ubiquitin ligase,” “ubiquitin-protein ligase,” and “E3 ligase”) refers to enzymes falling within Enzyme Commission/Classification (“EC”) 6.3.2.1. These enzymes function to recruit an E2 ubiquitin-conjugating enzyme that has been loaded with ubiquitin, recognizes a protein substrate, and assists or directly catalyzes the transfer of ubiquitin from the E2 to the protein substrate. The three substrates of an E3 ligase are ATP, ubiquitin, and a lysine residue on a protein; the three products of the reaction are AMP, diphosphate, and protein N-ubiquityl-lysine. Canonical ubiquitylation creates an isopeptide bond between a lysine residue on a target protein and the ubiquitin C-terminal Gly. In humans, E3 ligases regulate homeostasis, cell cycle, and DNA repair pathways. As a result, defects in certain E3 ligases have been implicated in a variety of cancers, including MDM2, BRCA1, and Von Hippel-Lindau tumor suppressor.

Fluorescence intensity measurements in the assays described above can be performed in any fluorimeter equipped with appropriate excitation and emission filters for FRET or quenched fluorescence measurements. In some embodiments the measurements are performed in 384-well microplates a Synergy 2 microplate reader (BioTek). In other embodiments the assays may be implemented in any fluorescence plate reader in 96-well, 384-well, 1536-well microplates or any other assay format.

### IV. FLUORESCENT RESONANCE ENERGY TRANSFER (FRET)/FLUORESCENCE QUENCHING

As used herein, the terms “FRET,” “fluorescence resonance energy transfer,” “Forster resonance energy transfer,” “fluorescence energy transfer,” and “resonance energy transfer” are used interchangeably, and refer to a nonradiative energy transfer process that occurs between two fluorophores or chromophores. The FRET phenomenon is when two fluorophores or chromophores are located as small distance (about 100 Å or shorter) from one another, and a fluorescence spectrum of one of the two fluorophores or chromophores (the donor) and an excitation spectrum of the other fluorophore or chromophore (the acceptor) overlap with each other, irradiation with a light of the excitation wavelength of the donor increases the fluorescence of the acceptor.

As used herein, a “fluorophore” or “chromophore” is a molecule that includes a region that adsorbs certain wavelengths of light. Fluorophores suitable for use in a FRET assay are known to the skilled person and are readily available. In one embodiment, a fluorophore may be a donor (also referred to as a donor probe and donor fluorophore). A donor probe refers to a molecule that will absorb energy and then re-emit at least a portion of the energy over time. In one embodiment, a fluorophore may be an acceptor (also referred to as an acceptor probe and acceptor fluorophore). An acceptor probe refers to a molecule that will accept energy non-radiatively from a donor, thus decreasing the donor's emission intensity and excited-state lifetime. A donor probe and acceptor probe that interact in this way are referred to herein interchangeably as a donor-acceptor pair or a FRET pair. In one embodiment an acceptor fluorophore may be a fluorescence quencher. A fluorescence quencher is a fluorophore that dissipates the energy transferred from a donor fluorophore non-radiatively into heat.

Examples of fluorophores that may be used as donors or acceptors in FRET pairs include, but are not limited to: 4-acetamido-4′-isothiocyanatostilbene-2,2′disulfonic acid acridine and derivatives such as acridine isothiocyanate, 5-(2′-aminoethyl)aminonaphthalene-1-sulfonic acid (EDANS), 4-amino-N-[3-vinylsulfonyl)phenyl]naphthalimide-3, 5disulfonate (LuciferYellow VS), N-(4-anilino-1-naphthyl)maleimide, Anthranilamide, Brilliant Yellow; coumarin and derivatives such as 7-amino-4-methylcoumarin (AMC, Coumarin 120), 7-amino-4-trifluoromethylcoumarin (Coumarin 151), cyanosine, 4′-6-diaminidino-2-phenylindole (DAPI), 5′,5″-dibromopyrogallol-sulfonephthalein (Bromopyrogallol Red), 7-diethylamino-3-(4′-isothiocyanatophenyl)-4-methylcoumarin, 4-(4′-diisothiocyanatodihydro-stilbene-2,2′-disulfonic acid, 4,4′-diisothiocyanatostilbene-2,2′-disulfonic acid, 5-[dimethylamino]naphthalene-1-sulfonyl chloride (DNS, dansyl chloride), 4-; eosin and derivatives such as eosin isothiocyanate; erythrosin and derivatives such as erythrosin B, erythrosin isothiocyanate; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein isothiocyanate, fluorescamine (only fluorescent when it reacts with primary amines), IR144, IR1446, Malachite Green isothiocyanate, 4-methylumbelliferone, pararosaniline, Phenol Red, B-phycoerythrin, o-phthaldialdehyde (only fluorescent when it reacts with primary amines); pyrene and derivatives such as pyrene butyrate, succinimidyl 1-pyrene butyrate, Reactive Red 4 (Cibacron® Brilliant Red 3B-A); rhodamine and derivatives such as 6-carboxy-X-rhodamine (ROX), 6-carboxyrhodamine (R6G), lissamine rhodamine B sulfonyl chloride, rhodamine B, rhodamine 123, rhodamine X isothiocyanate, sulforhodamine B, sulforhodamine 101, sulfonyl chloride derivative of sulforhodamine 101 (Texas Red), N,N,N′,N′-tetramethyl-6-carboxyrhodamine (TAMRA), tetramethyl rhodamine, tetramethyl rhodamine isothiocyanate (TRITC), riboflavin, BODIPY dyes, AlexaFluor™ dyes, both of which are made available from Invitrogen, CA, Cyanine dyes (Cy™3, Cy™3B, Cy™5, Cy™7) made available from GE HealthCare Life-Sciences. Some exemplary dyes that may be used as acceptors only include, but not limited to, 4-(4′-dimethylaminophenylazo)benzoic acid (DABCYL) and derivatives, Cy™5Q, and Cy™7Q Examples of AlexaFluor dyes include, but are not limited to: Alexa Fluor® 350, Alexa Fluor® 405, Alexa Fluor® 430, Alexa Fluor® 488, Alexa Fluor® 500, Alexa Fluor® 514, Alexa Fluor® 532, Alexa Fluor® 546, Alexa Fluor® 555, Alexa Fluor® 568, Alexa Fluor® 594, Alexa Fluor® 610, Alexa Fluor® 633, Alexa Fluor® 635, Alexa Fluor® 647, Alexa Fluor® 660, Alexa Fluor® 680, Alexa Fluor® 700, Alexa Fluor® 750, Alexa Fluor® 790, and the like.

In certain aspects one of the energy transfer pair can be a quencher. Fluorescent quenching refers to any process that decreases the fluorescence of a molecule, such as black hole quenchers (BHQ) commercially available from Biosearch Technologies. Fluorescent quenchers include, but are not limited to, 4-(4′-dimethylaminophenylazo)benzoic acid) (DABCYL), DABSYL, QSY-7, QSY-33, BHQ0, BHQ1, BHQ3, BHQ4, Iowa Black™ (Integrated DNA Tech Coralville Iowa, BlackBerry™ Quencher 650 (Berry & Assoc Dexter, MI), cyanine dyes (including nitrothiazole blue (NTB)), anthraquinone, malachite green, nitrothiazole, and nitroimidazole compounds. Different quencher dyes are suitable for use with specific fluorophores, including FAM, TET, JOE, HEX, Oregon Green®, TAMRA, ROX, Cyanine-3, Cyanine-3.5, Cyanine-5 and Cyanine-5.5 (e.g., CY-3, CY-5, CY-3.5, CY5.5, etc).

### V. KITS AND COMBINATIONS OF REAGENTS

Some uses of FRET-active E2-Ub/Ubl conjugates described herein may need E2-Ub/Ubl conjugates to be generated by researchers as part of their research and development activities. Another aspect of the present invention provides kits and combinations of reagents (e.g., Ub-fluorophore and/or E2-Fluorophore) for facilitating research and development activities involving FRET-active E2-Ub/Ubl conjugates or reagents for their production. In an embodiment, the kit comprises at least two reagents: a Ub/Ubl (ubiquitin or ubiquitin-like protein) labeled with one fluorophore as described above (or the reagents and substrates for producing the same) and a E2 protein labeled with a second fluorophore (or the reagents and substrates for producing the same), such that the first and the second fluorophores represent a FRET-active pair. The kit may optionally include one or more reaction buffers or washing buffers, an instruction for assay procedure, one or more E1 ubiquitin activating enzyme(s) or one or more E3 ubiquitin ligases.

In another aspect, the present invention provides kits for performing FRET or quenched fluorescence assays of E1 activity. For example, the kit can include a Ub/Ubl (ubiquitin or ubiquitin-like protein) labeled with one fluorophore as described above and a E2 protein labeled with a second fluorophore, such that the first and the second fluorophores represent a FRET-active pair, buffers containing other reagents needed for synthesis of the FRET-active E2-Ub/Ubl conjugate catalyzed by an E1 ubiquitin activating enzyme. Optionally, the kit can also include one of more E1 ubiquitin activating enzyme to be used as a positive control or an inactive E1 variant to be used as a negative control. Optionally, the kit can also include apyrase or another enzyme for hydrolyzing ATP and thus stopping variation ATP dependent reaction(s).

In another aspect, the present invention provides kits for performing FRET or quenched fluorescence ubiquitin discharge assays of E3 ubiquitin ligase activity. In one embodiment, the kit comprises reagents for performing the assays. For example, the kit can include a Ub/Ubl (ubiquitin or ubiquitin-like protein) labeled with one flurophore as described above and a E2 protein labeled with a second fluorophoe, such that the first and the second fluorophores represent a FRET-active pair, one or more E1 ubiquitin activating enzyme to catalyze synthesis of the the FRET-active E2-Ubl conjugate, buffers containing other reagens needed for E1 activity. Optionally, the kit can also include apyrase or another enzyme for hydrolyzing ATP and thus stopping the reaction. Optionally, the kit can also include one of more E3 ubiquitin ligase to be used as a positive control or an inactive E3 variant to be used as a negative control.

The kit can also comprise a washing solution or instructions for making a washing solution.

In a further embodiment, such a kit can comprise instructions for suitable operational parameters and/or assay performance. For example, the instructions may inform a consumer about how to collect the sample, how to prepare a conjugate and/or enzyme to be detected.

In yet another embodiment, the kit can comprise one or more containers with reagents.

The reaction buffer may include reagents like salts, solvents, buffers, neutral proteins, e.g., albumin, detergents, etc. The reaction buffer facilitates optimal ubiquitination enzyme activity and/or reduce non-specific or background interactions. The reaction buffer may also include reagents that otherwise improve the efficiency of the assay, such as protease inhibitors, nuclease inhibitors, anti-microbial agents, etc. In a preferred embodiment, the reaction buffer includes adenosine tri-phosphate (ATP). In another embodiment, the kit includes apyrase or another enzyme for depletion of ATP.

The kits are not limited to the embodiments described above, but include other combinations of reagents whose primary use, explicit or implicit, is for the production of FRET-active E2-Ubl conjugates described in this invention.

### VI. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### How Proximity of Three Ring Domains Mediates Autoubiquitination of Trim5Alpha

The novel reagents and methods described herein were used, as an example, to investigate TRIM5alpha, a member of the TRIM sub-family of a large family of RING E3 ubiquitin ligases, that forms part of the innate antiviral immune defenses. TRIM5a is one of the most extensively studied members of the TRIM family, owing to its role in determining host tropism of primate immunodeficiency viruses, most notably—HIV (Stremlau et al., Nature 427, 848-53, 2004; Sayah et al., Nature 430, 569-73, 2004; Ganser-Pornillos and Pornillos, Nat Rev Microbiol 17, 546-56, 2019). The C-terminal SPRY domain of TRIM5a has evolved to bind retroviral capsids and its high variability among primates is thought to reflect the ongoing evolutionary antagonism between primate immunodeficiency viruses and their hosts (Sebastian and Luban, Retrovirology 2, 40, 2005; Stremlau et al., Proc Natl Acad Sci USA 103, 5514-9, 2006; Diaz-Griffero et al., Virology 351, 404-19, 2006; Ohkura et al., Journal of Virology 80, 8554-65, 2006; Song et al., Journal of Virology 79, 6111-21, 2005; Sawyer et al., Proc Natl Acad Sci USA 102, 2832-7, 2005; Stremlau et al., J Virol 79, 3139-45, 2005). The mechanism of the TRIM5α-mediated block to retroviral infectivity is not fully understood, but it is well established that it depends on direct binding of TRIM5α to mature retroviral capsids upon their entry into the cytoplasm of the target cell (Sebastian and Luban, Retrovirology 2, 40, 2005; Stremlau et al., Proc Natl Acad Sci USA 103, 5514-9, 2006). The capsid is not the ubiquitination substrate of this E3 ligase. Instead, TRIM5α is thought to undergo autoubiquitination upon binding to the capsid. The novel methods and reagents described herein were used by us to investigate how association of TRIM5α with the capsid promotes its autoubiquitination.

TRIM5alpha (NP_149023.2) is an E3 ubiquitin ligase of the TRIM family that binds to the capsids of primate immunodeficiency viruses and blocks viral replication after cell entry. Synthesis of K63-linked polyubiquitin catalyzed by TRIM5alpha is thought to contribute to the antiviral function of the protein, but the mechanism that couples capsid binding to E3 activation remains poorly understood. In this study we delineate how polyubiquitin synthesis is upregulated by transient proximity of three RING domains in honeycomb-like assemblies formed by TRIM5alpha on the surface of the retroviral capsid. Proximity of three RINGs creates an asymmetric arrangement, in which two RINGs form a catalytic dimer that activates E2-ubiquitin conjugates and the disordered N-terminus of the third RING acts as the substrate for N-terminal auto-ubiquitination. RING dimerization is required for activation of the E2s that contribute to the antiviral function of TRIM5alpha, UBE2W and UBE2N/V2, whereas the proximity of the third RING enhances the rate of each of the two distinct steps in the autoubiquitination process: the N-terminal mono-ubiquitination of TRIM5alpha by UBE2W and the subsequent extension of the K63-linked polyubiquitin chain by UBE2N/V2. The mechanism we describe explains how recognition of infection-associated epitope patterns by TRIM proteins triggers polyubiquitin-mediated downstream events in innate immunity.

E3 ubiquitin ligases from the TRIM family are increasingly recognized for their diverse contributions to innate immune defenses (van Gent et al., Annu Rev Virol 5, 385-405, 2018), but mechanisms that promote polyubiquitin synthesis by TRIM proteins and activate polyubiquitin-dependent immune responses, remain poorly understood. For example, TRIM5alpha-mediated restriction of retroviral replication depends on direct association of TRIM5alpha with the retroviral capsid, but the capsid is not the ubiquitination substrate of this E3 ligase. Instead, TRIM5α is thought to undergo autoubiquitination upon binding to the capsid, and here we investigate how association of TRIM5α with the capsid promotes its autoubiquitination.

The mechanism of TRIM5alpha E3 activation depends on proximity of three RING domains. At the vertices of the honeycomb-like assemblies formed by TRIM5α on the capsid surface, three RING domains from three distinct TRIM5alpha dimers are brought together by the interactions mediated by the B-box domains (Ganser-Pornillos and Pornillos, Nat Rev Microbiol 17, 546-56, 2019). Proximity of three RINGs leads to an asymmetric arrangement in which two RING domains associate into a compact dimer, whereas the third RING remains in a monomer-like state. The asymmetry of this arrangement arises from the disorder-to-order transitions in the polypeptide segments flanking the Zn-binding core of the RING domain, which are unstructured and highly mobile in the RING monomer, but fold into a well-defined amphipathic four-helix bundle in the dimer.

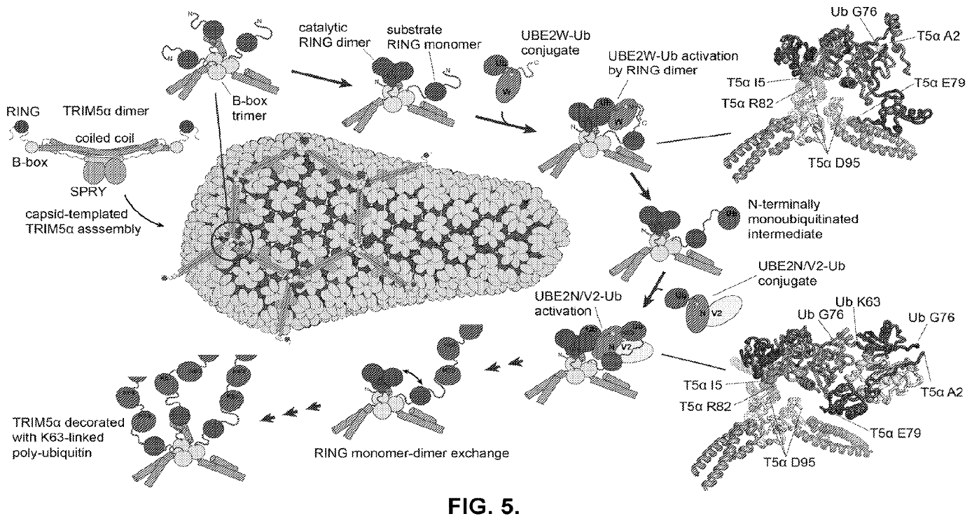

The wealth of structural data on different TRIM5alpha domains, TRIM5alpha-capsid interactions and RING-mediated recruitment and activation of E2-Ub conjugates makes it possible to model the structural basis for our observations with high confidence (FIG. 5). The disorder-to-order transitions and the asymmetry of the RING trimer enable the distinct functional contributions of the three RINGs to the autoubiquitination process. The RING dimer performs a catalytic role by stabilizing a particular relative orientation of E2 and ubiquitin in the E2-ubiquitin conjugate, in which the thioester bond is exposed and primed for ubiquitin transfer. Here this mechanism contributes to the activation of the two E2s known to contribute to the antiviral function of TRIM5α-UBE2W and the heterodimeric UBE2N/V2. Structural constraints prevent RING dimers from catalyzing transfer of the ubiquitin onto itself because the thioester bond in the active site of the E2-ubiquitin conjugate points away from the E2-RING interface. The asymmetry of the RING trimer therefore reflects the inherent functional asymmetry in the process of RING autoubiquitination, in which two RINGs perform a catalytic role and the third RING acts as the ubiquitination substrate.

The proximity of the third RING and the mobility of its backbone are likely to facilitate the autoubiquitination process in several ways. Long and mobile N-termini in proteins are known to be the preferred ubiquitination substrates for UBE2W (Vittal et al., Nat Chem Biol 11, 83-9, 2015). In addition, the backbone flexibility may further enhance autoubiquitination by extending the reach of the third RING and increasing the rate at which its N-terminus encounters the active site of the UBE2W-Ub conjugate recruited and activated by the RING dimer. Proximity and backbone flexibility elevate the effective concentration of the preferred substrate in the vicinity of the activated UBE2W-Ub conjugate and make the N-terminus of the third RING the predominant ubiquitination site that outcompetes other potential substrates. Once the third RING is monoubiquitinated, the same mechanism greatly favors the K63 residue of the N-terminally attached ubiquitin as the preferred site for ubiquitin discharge from the UBE2N/V2-Ub conjugate. This explains the strong enhancement of ubiquitin discharge and the change in the products of UBE2N/V2-mediated polyubiquitin synthesis that we observe with the monoubiquitinated trimeric R3 WT construct. The question whether the unconjugated or conjugated K63-linked polyubiquitin represents the functionally important signaling species has been controversial in the studies of RIG-I signaling (Wang and Hur, Semin Cell Dev Biol 111, 76-85, 2021). Our findings indicate that the TRIM5alpha-attached, K63-linked polyubiquitin (rather than unconjugated polyubiquitin chains) is the functionally important product that mediates downstream events promoted by the E3 activity of TRIM5alpha assembled on the capsid surface. In addition, we present evidence that RING monomers can swap their roles within the RING trimer, which suggests that all three RINGs are eventually modified and that the vertices of the honeycomb-like TRIM5alpha assemblies become decorated with three polyubiquitin chains.

Finally, the requirement for proximity of three RING domains as the minimum number needed for efficient autoubiquitination offers insight into how TRIM proteins may recognize particular three-dimensional patterns of binding epitopes. Self-association of isolated TRIM5alpha B-box domains in vitro is known to be relatively weak and the assembly of full-length TRIM5alpha dimers on the capsid surface is thought to be enhanced by the avidity effect arising from the interaction of the SPRY domains with multiple distinct binding epitopes on the capsid surface (Wagner et al., Elife 5, 2016; Wagner et al., J Virol 92, 2018). The cooperativity between B-box association and SPRY-capsid interactions is expected to be strongly dependent on the distance between SPRY binding epitopes, as dictated by the length of the coiled-coil rods, and the angles between them, as dictated by the preferred geometry of B-box self-association. The reversible self-association of the mobile RING segments into a four-helix bundle of the RING dimer described here is likely to provide an additional contribution to the capsid binding avidity and the cooperativity of the TRIM5apha assembly/disassembly process. As a result, proximity of three RING domains depends on binding of at least three distinct TRIM5alpha dimers to a particular spatial arrangement of three distinct sets of epitopes recognized by the SPRY domains. Furthermore, because the capsid-templated TRIM5alpha assembly/disassembly process is highly cooperative, formation of a larger honeycomb segment may be required to ensure that RING trimers persist long enough to complete synthesis of multiple polyubiquitin chains at the vertices of the honeycomb. In summary, the cooperativity between ligand binding mediated by the C-terminal domains and self-association mediated by the B-box and RING domains of TRIM proteins ensures that foci of K63-linked polyubiquitin chains are only synthesized when multiple TRIM binding epitopes are arranged in a particular pattern in three dimensions.

Conserved domain architecture of the TRIM family suggests that the mechanism linking RING oligomerization to autoubiquitination described here is conserved in one form or another across the TRIM family. Indeed, structural and functional studies on TRIM21 and TRIM25 revealed numerous functional similarities between these TRIM family members suggesting that the mechanisms controlling their E3 activity are closely related. Although the exact role of K63-linked polyubiquitin in inducing antiviral immune responses is not fully understood, K63-linked polyubiquitin chains are thought to promote assembly and activation of various multiprotein complexes mediating downstream immune events. Collectively, results reported here and in other recent studies on TRIM5α and related E3 ligases shed light on the ability of TRIM proteins to detect particular patterns of multiple binding epitopes and to create immunostimulatory foci of K63-linked polyubiquitin either through autoubiquitination, or through ubiquitination of other substrates.

### A. Results

Recombinant constructs were designed to mimic distinct RING oligomerization states of TRIM5α. The inventors generated a series of recombinant RING constructs that mimic different relative arrangements of RING domains—monomer, dimer and trimer—thought to occur in the distinct oligomerization states of TRIM5α.

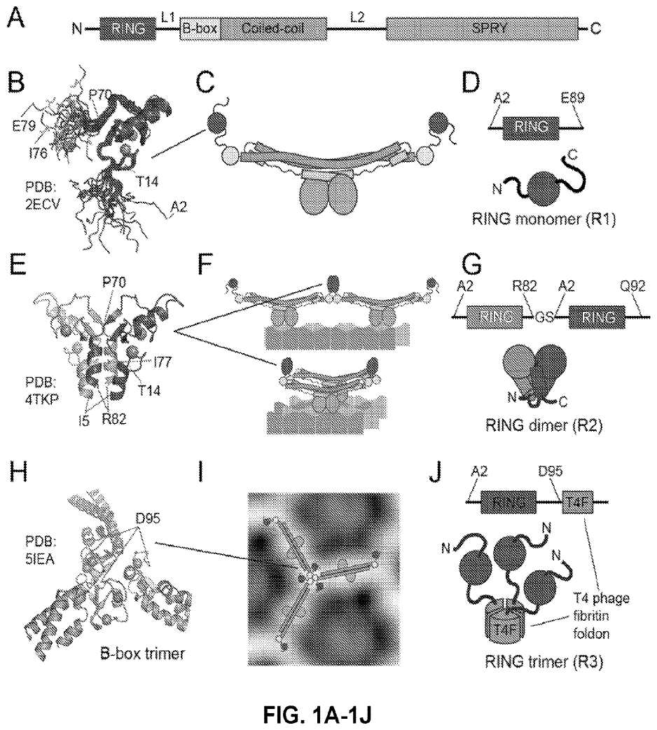

The isolated TRIM5α RING domain is predominantly monomeric at low concentrations³⁶, and NMR studies revealed that the long N-terminal and C-terminal segments of the RING domain, which flank the Zn-coordinating core, are largely unstructured in solution (PDB: 2ECV; FIG. 1). In contrast, once crystallized, TRIM5α RING domains were found to form dimers held together by an extensive four-helix-bundle interface (FIG. 1C). This dimerization mode, in which the unstructured N-terminal and C-terminal segments fold into two amphipathic alpha helices and associate into a four-helix bundle with hydrophobic interior, is highly conserved in the TRIM family as it has now been observed in all known crystal structures of TRIM RINGs (TRIM5α: 4TKP, TRIM37: 3LRQ, TRIM69: 6YXE, TRIM23: 5VZW, TRIM32: 5FEY, TRIM25: 5EYA, TRIM21: 6S53 and possibly others).

To gain further insight into transient RING dimerization we took advantage of the two constructs used in our previous work, an isolated RING monomer and a tandem RING dimer, denoted as R1 and R2 here. The R1 construct encompasses residues 2-89 of the rhesus TRIM5α. This construct contains all residues needed for RING dimerization but remains predominantly monomeric at low concentrations in solution. The monomeric state of this construct is likely to represent the predominant conformational state of the RING domain within isolated TRIM5α dimers, because the two RINGs are held almost 20 nm apart on the opposing ends of the antiparallel coiled coil and cannot dimerize within the TRIM5α dimer (FIG. 1). Upon dimerizaton the N- and C-termini of the two monomers come into proximity, and RING dimers are commonly stabilized by creating a tandem RING construct with a short linker connecting the C-terminus of the first RING to the N-terminus of the second. We used this strategy to prepare a tandem construct, R2, in which residue R82 of the first RING is linked to residue A2 of the second through a dipeptide Gly-Ser linker (FIG. 1C).

Finally, to mimic proximity of three RING domains promoted by B-box trimerization at the vertices of the honeycomb-like TRIM5α assemblies we prepared the R3 construct, in which the RING domain (residues 1-95) is fused at the C-terminus to the T4 fibritin foldon, a 27-residue peptide that forms tight trimers and is often used to create or stabilize trimeric proteins (FIG. 1D). In the T4 foldon trimer the N-termini of each monomer are located on one face of the trimer approximately 12 Å from each other, which makes it a reasonably good mimic of the B-box trimer, whose N-termini (residue D95) are located 22 Å angstroms apart in the crystal structure. The unstructured linker between RING and B-box domains (residues 83-95) is sufficiently long to make differences in the spacing between N-termini of the T4 foldon and B-box trimer insignificant.

Previously we investigated functional importance of RING dimerization by introducing a point mutation (I77R in the rhesus monkey TRIM5α) that replaces an isoleucine residue in the interior of the four-helix bundle of the dimer to an arginine (I76, in the human TRIM5α). This mutation is expected to disrupt the four-helix bundle and RING dimerization, but to have a minor effect on the monomeric form of the RING domain, in which residues following residue P70 are unstructured (FIG. 1A and FIG. 1). In this study we introduced the I77R mutation into the R1 and R3 constructs. In summary, the five RING domain constructs (R1-WT, R1-177R, R2-WT, R3-WT and R3-177R) were investigated here to establish the structural and biochemical determinants of TRIM5α E3 activity.

Proximity of three RINGs creates an asymmetric arrangement, in which two RINGs form a compact dimer and the third remains in a monomer-like state with an unstructured N-terminal segment. Recombinant RING constructs were first investigated by sedimentation velocity analytical ultracentrifugation to ascertain that they form the desired oligomeric states. R2-WT and R3-WT constructs sedimented at the rates that were close to the theoretically predicted dimer and trimer values, respectively. Sedimentation velocity analysis of the R1-WT sample revealed that the sample exists as a mixture of the monomer and the dimer. In contrast, no detectable dimer component was present in the R1-177R sample, as expected. To gain further insight into the monomer-dimer equilibrium of the R1-WT samples, we carried out series of sedimentation equilibrium experiments performed at different protein concentrations and rotor speeds. Fitting the entire dataset using the simple monomer-dimer equilibrium model yielded the dimerization constant K_{D} of approximately 60 μM with good residuals except in the lowest concentration samples.

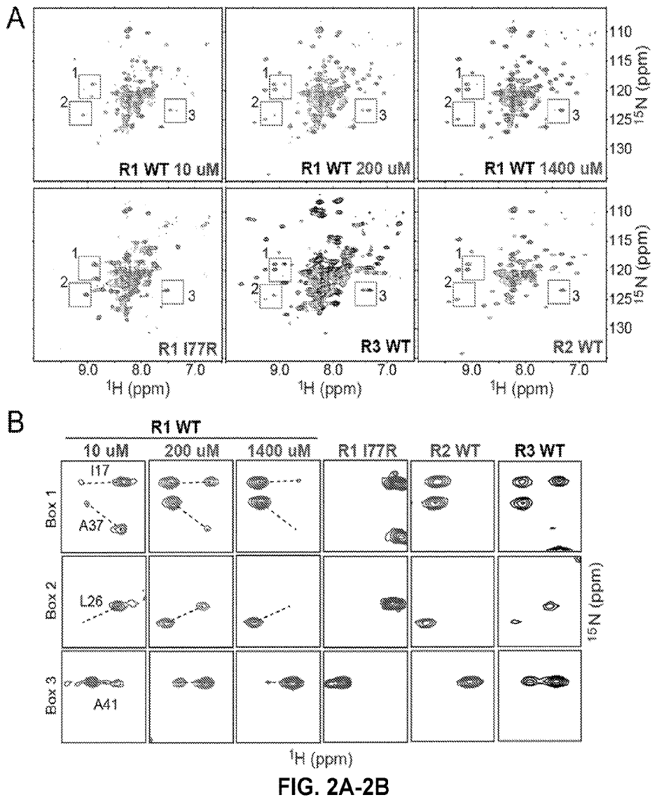

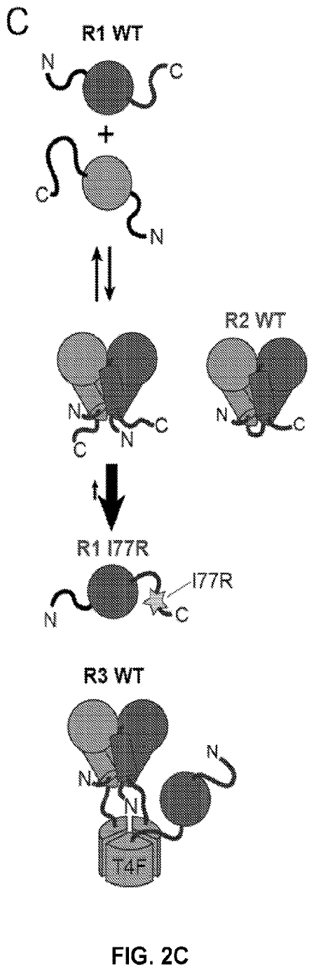

We then investigated RING conformational states in different constructs by NMR spectroscopy. ¹⁵N HSQC spectra of R1-WT, R2-WT and R1-177R samples collected at high protein concentration revealed that the R1-WT and R2-WT spectra were remarkably similar to each other with most peaks displaying a near perfect overlap (FIG. 2A). In contrast, the R1-177R spectrum was dramatically different with significantly lower overall dispersion and higher signal crowding in the central area of the spectrum, which is indicative of significantly higher disorder present in the R1-177R sample compared to R1-WT and R2-WT. We then collected series of ¹⁵N HSQC spectra by gradually decreasing protein concentration of the R1-WT sample from 1400 M to 10 μM. As the concentration of the sample was lowered one set of peaks became progressively weaker whereas a new set of peaks appeared and became dominant (FIG. 2A and FIG. 2B). At 10 μM the R1-WT spectrum became strikingly similar to that of the R1-177R sample. The findings confirmed that the R1-WT construct exists in a dimer-monomer equilibrium and that the two distinct sets of NMR signals present at high and low concentrations correspond to the dimeric and monomeric states of the protein, respectively. In addition, the spectra revealed that the I77R mutation does not significantly perturb the structure of the monomeric state of R1-WT, in which the N-terminal and C-terminal segments are largely disordered. The dimer-monomer exchange rate is slow on the NMR time scale (<10 Hz) which indicates that the dimer association rate is slow in agreement with a major conformational change taking place upon dimerization. Finally, the spectra of the R3-WT sample contained both sets of NMR signals, corresponding to the monomer and the dimer, which revealed that when three RING domains are brough together, two RINGs form the dimer observed in the crystal structure, whereas the third RING remains in a monomer-like state with an unstructured N-terminal segment (FIG. 2C).

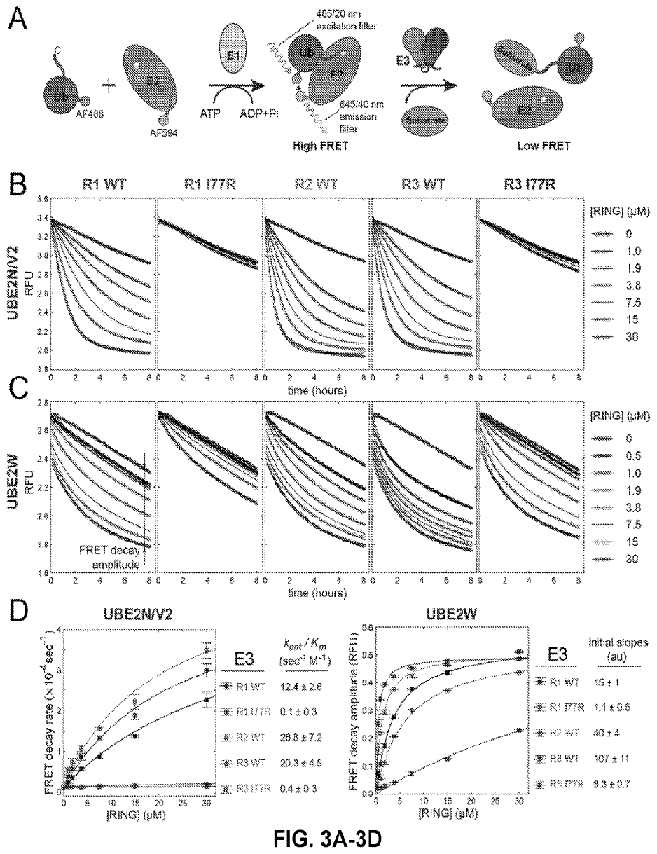

A FRET-based ubiquitin discharge assay reveals that RING dimerization is required for activation of UBE2N V2-Ub and UBE2W-Ub conjugates for ubiquitin transfer. To evaluate contributions of distinct RING oligomeric states to TRIM5α autoubiquitination we investigated the catalytic activity of the RING constructs. E3 ubiquitin ligases catalyze the transfer of ubiquitin from the high-energy thioester-linked E2-Ub conjugates onto an amine group of the substrate. One of the challenges in the mechanistic studies of E3 ligases is the difficulty of measuring rates of chemical transformations catalyzed by E3 ligases with sufficient precision. In this study we developed a FRET-based ubiquitin discharge assay that allows continuous, real-time measurement of E3-catalyzed ubiquitin release from E2-Ub conjugates present at nanomolar concentrations in multiple samples in parallel using a fluorescence plate reader (FIG. 3A). To this end, different E2s and ubiquitin were N-terminally labeled with acceptor (AF594) and donor (AF488) fluorescent dyes, respectively, using sortase-catalyzed transpeptidation. AF594-E2s were conjugated to AF488-ubiquitin using recombinant E1 enzyme in a standard procedure and the conjugation reactions were quenched with apyrase (Pickart and Raasi, Methods Enzymol 399, 21-36, 2005). FRET signals of the E2-Ub conjugates were recorded as a function of time following addition of different RING constructs using AF488 excitation (485/20 nm) and AF594 emission (645/40 nm) bandpass filters. We observed that more than 40% of the total fluorescence signal decayed in a RING-dependent fashion and could, therefore, be attributed to the FRET signal of the conjugate. The outstanding sensitivity, signal-to-noise and throughput of the FRET decay measurements enabled robust and accurate quantification of ubiquitin discharge.

We first investigated ubiquitin discharge from the UBE2N/V2-Ub conjugate catalyzed by different TRIM5α RING constructs (FIG. 3B). Ubiquitin discharge rates were determined by fitting the FRET decay curves with a simple exponential decay function (Methods). Subsequently, dependence of discharge rates on the concentration of RING monomers in the sample was modelled with the hyperbolic Michaelis-Menten equation and the k_{cat}/Kₘ values (initial slopes of the Michaelis-Menten curves) were used to quantify the E3 activity of each RING construct (FIG. 3D). In agreement with previous work (Yudina et al., Cell Rep 12, 788-97, 2015), we find that catalysis of ubiquitin discharge from the UBE2N/V2-Ub conjugate is virtually abolished by the I77R mutation in the R1-177R and R3-177R constructs. The E3 activity of the R2-WT is also significantly higher than that of the R1-WT. These observations confirm that RING dimerization promotes ubiquitin discharge from the UBE2N/V2-Ub conjugate by stabilizing a particular relative arrangement of UBE2N and donor ubiquitin, in which the high-energy thioester bond is exposed and primed for nucleophilic attack by the K63 amine of the substrate ubiquitin. We also observe that the E3 activity per RING monomer is lower in the R3-WT construct than in R2-WT. This observation is in agreement with our NMR data, which indicate that in the R3-WT sample a portion of RING units remain in the monomer-like, catalytically inactive conformation.

We then used the FRET assay to investigate RING-catalyzed ubiquitin discharge from UBE2W, another E2 known to mediate the antiviral activity of TRIM5α. Kinetics of FRET decay observed in these experiments was notably different from what was observed for UBE2N/V2. The initial rapid burst of FRET decay was followed by a slower decay phase, the amplitude of which, but not the rate, was dependent on the RING concentration (FIG. 3C). Although the mechanistic basis of this distinctive kinetics remains unknown and will require further study, the dataset offers another illustration of the power of the FRET-based ubiquitin discharge assay for studies of E3 kinetics. Because the rate of the initial burst, albeit RING concentration dependent, was too fast to be measured accurately, the amplitude of the RING-dependent FRET decay was used instead. Dependence of decay amplitude on RING concentration displayed a good fit to the Michaelis-Menten equation, and the initial slopes of the Michaelis-Menten curves were once again used as a quantitative measure of the E3 activity. This dataset revealed that albeit the discharge from the UBE2W-Ub conjugate was less sensitive to the I77R mutation, the R3-177R and R1-177R constructs retained less than 10% of the R3-WT and R1-WT E3 activity, respectively. These data establish that akin to UBE2D and UBE2N/V2, UBE2W is another E2 whose ability to transfer ubiquitin onto its substrates is strongly enhanced by RING dimerization. Another notable difference with the UBE2N/V2 dataset was the significantly higher activity of the R3-WT construct in comparison to R2-WT. We hypothesized that this enhancement resulted from the third RING in the R3-WT construct acting as the substrate for ubiquitin transfer and proceeded to a more careful examination of the products of the ubiquitination reactions catalyzed by our engineered RING constructs.

Proximity of the third RING with an unstructured N terminus facilitates N-terminal monoubiquitination of TRIM5α by UBE2W and subsequent elongation of a TRIM5α-attached K63-linked polyubiquitin chain by UBE2N V2. UBE2W is a specialized E2 conjugating enzyme that displays a strong preference for flexible N-termini in proteins as ubiquitination substrates. One peculiar structural feature of ubiquitin is that its N-terminal methionine forms an integral part of the protein structure and, as a result, cannot be modified by UBE2W. The typical product of UBE2W-mediated protein ubiquitination is, therefore, the N-terminally monoubiquitinated substrate protein, and the N-terminally monoubiquitinated TRIM5α was shown to be the dominant product of UBE2W-dependent autoubiquitination of TRIM5α in vitro and cells.

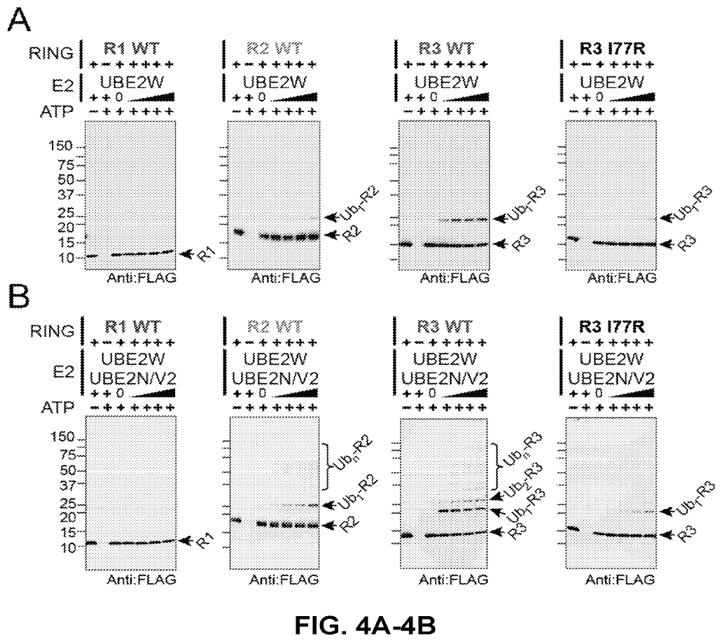

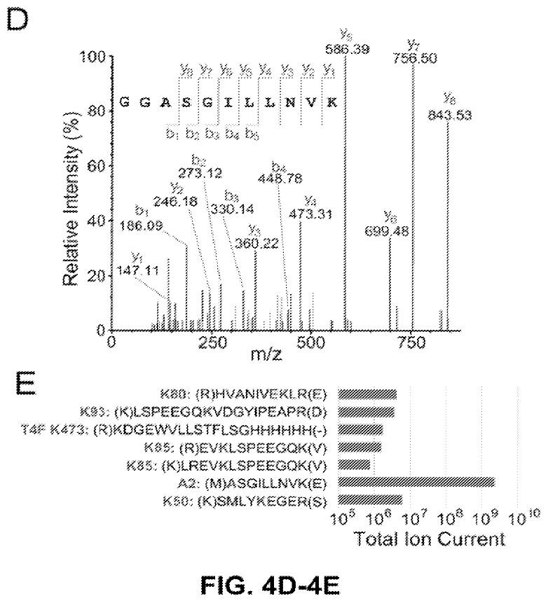

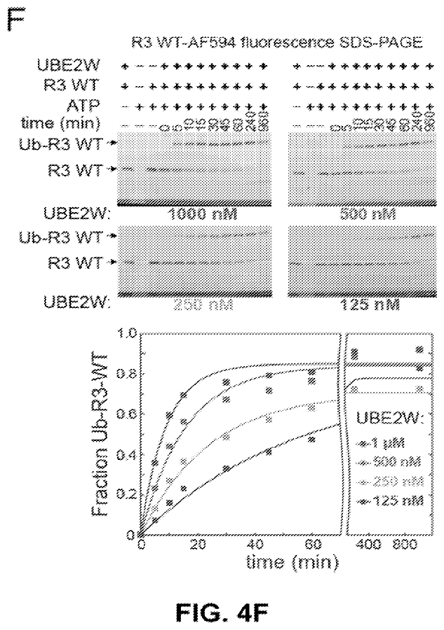

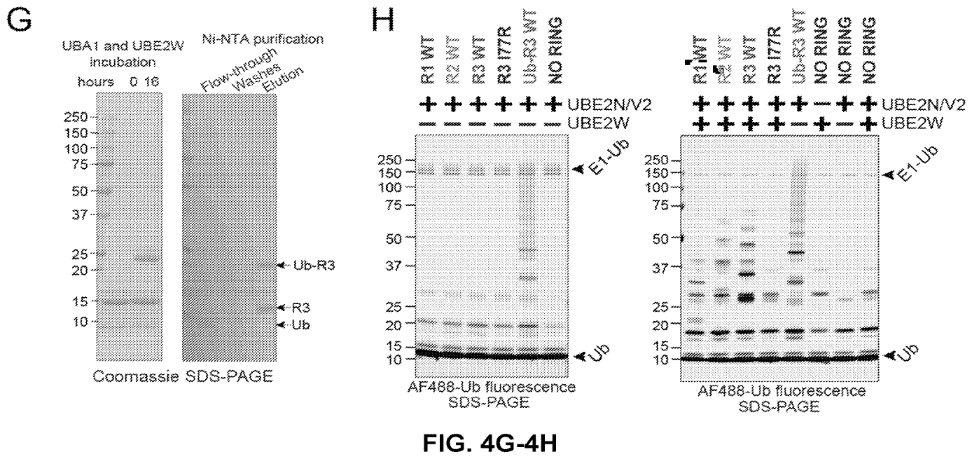

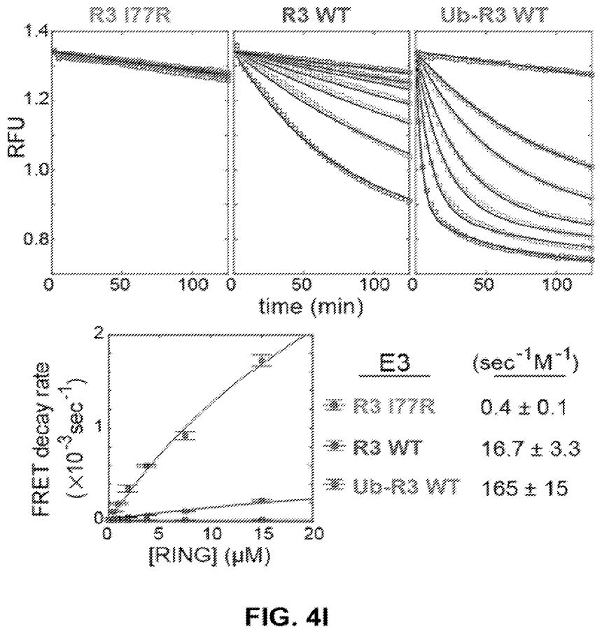

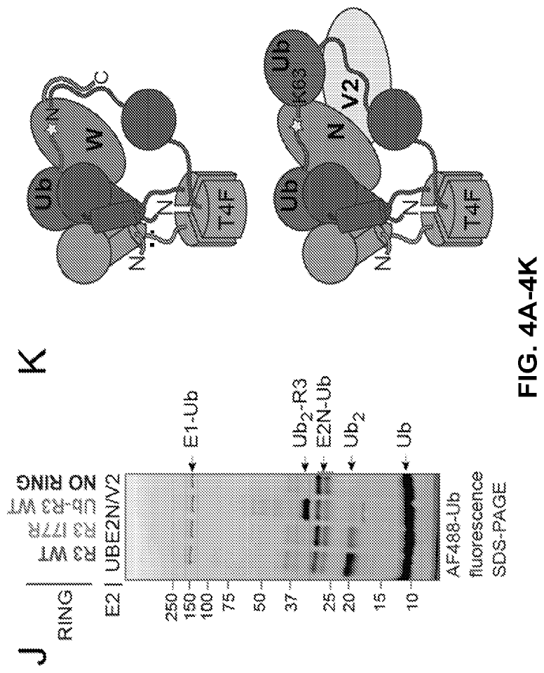

We first investigated ubiquitination products generated by the RING constructs using Western blot (FIG. 4A and FIG. 4B). A C-terminal FLAG tag was fused to the RING constructs and used to visualize products formed in conventional in-vitro ubiquitination reactions containing E1, different E2 combinations, ubiquitin and other required components. In agreement with previous work (Fletcher et al., EMBO J 34, 2078-95, 2015; Fletcher et al., Cell Host Microbe 24, 761-775 e6, 2018), when UBE2W was the only E2 included in the reaction, a single additional band corresponding to monoubiquitylated RING construct was the predominant product of the ubiquitination reaction (FIG. 4A). Although some RING monoubiquitination could be observed in all reactions, modification of the R3-WT construct was clearly the most efficient. In agreement with ubiquitin discharge assays, these observations also indicate that the UBE2W-dependent ubiquitination of the R3-WT construct is indeed intramolecular and that the long, disordered N-terminus of the third, monomer-like RING within the R3-WT construct is the preferred ubiquitination substrate. When UBE2N and UBE2V2 are included in these ubiquitination reactions together with UBE2W, we observe polyubiquitination of the TRIM5α RING constructs and once again the R3-WT is the most active among the tested constructs (FIG. 4B).

For a more direct and accurate comparison of products formed in different combinations of UBE2W, UBE2N/V2 and various RING constructs we performed all reactions in parallel and analyzed the products on a single SDS-PAGE gel by Western blotting with either anti-FLAG or anti-K63-linked-polyubiquitin antibodies (FIG. 4C). The anti-FLAG WB analysis confirmed that the R3-WT construct displayed most potent autoubiquitination activity. It also revealed that RING polyubiquitination by UBE2N/V2 requires prior monoubiquitination by UBE2W, in agreement with prior studies using different TRIM5α constructs. Another important effect of UBE2W-mediated monoubiquitination on UBE2N/V2-dependent products is evident from the WB with anti-K63 antibodies. UBE2N/V2 in known to have some background activity that results in the synthesis of unconjugated, K63-linked polyubiquitin chains in vitro, and we can detect this activity in our experiments. We observe that synthesis of unconjugated K63-linked polyubiquitin is strongly enhanced by all dimerization-competent RING constructs, R1-WT, R2-WT and R3-WT, but not by the R3-I77R mutant. Whereas inclusion of UBE2W had only a minor effect on the synthesis of unconjugated K63-linked polyubiquitin by the R1-WT and R2-WT constructs, the R3-WT construct displayed a notably different pattern of low molecular weight products and significantly denser staining at high molecular weights that overlapped with the anti-FLAG staining of polyubiquitinated R3-WT. These observations revealed that once the R3-WT trimer is monoubiquitinated by UBE2W a much larger fraction of total K63-linked polyubiquitin products generated by UBE2N/V2 is covalently attached to the RING rather than unconjugated.

Series of in-vitro and cellular experiments by Fletcher et al. strongly indicated that the N-terminus is the functionally relevant modification site of TRIM5α by UBE2W. We used mass spectrometry (MS) to verify that the robust UBE2W-mediated autoubiquitination of the R3-WT construct similarly favors the N-terminal attachment of ubiquitin. Indeed, the MS analysis revealed that modification of the N-terminal amine in R3-WT by UBE2W was preferred over other modification sites by more than two orders of magnitude as indicated by the corresponding ion currents (FIG. 4D and FIG. 4E).

We then asked whether the asymmetry of the trimeric R3-WT construct results in the asymmetry of the formed products, with only one out of three RINGs within the trimer modified with ubiquitin. To this end we labeled the R3-WT construct with a C-terminal fluorescent tag using sortase and quantified monoubiquitination stoichiometry as a function of time by directly imaging and integrating fluorescence intensity of the R3-WT and Ub-R3-WT bands in SDS-PAGE gels (FIG. 4F). This fluorescent SDS-PAGE analysis is immune to non-linearities associated with membrane transfer and antibody staining in WB. We observed that monoubiquitination of the R3-WT construct goes to completion with no apparent accumulation of intermediates at 1:3 stoichiometry. These observations suggest that the monomer-dimer exchange within the R3-WT construct enables monoubiquitination of all three RINGs by UBE2W.

Finally, to better establish how the initial monoubiquitination event alters UBE2N/V2-dependent activity and products we prepared a Ub-R3 WT sample by performing enzymatic monoubiquitination of the R3 WT construct using UBA1 and UBE2W and purifying the products. Because of vast excess of R3 WT over UBA1 and UBE2W used in these reactions the monoubiquitination did not go to completion and approximately half of the total RING domains were monoubiquitinated in the purified Ub-R3 WT sample (FIG. 4G). First, we compared the ability of the Ub-R3 WT sample and other RING constructs to catalyze polyubiquitin synthesis in the presence of UBE2N/V2. We performed ubiquitination reactions in the presence of fluorescently labeled ubiquitin and analyzed the products using direct fluorescent imaging of SDS-PAGE gels (FIG. 4G). These experiments revealed that the Ub-R3 WT sample is by far the most catalytically active. Even when UBE2W was included in the reactions with the unmodified R3 WT construct, its activity was significantly lower than that of the Ub-R3 WT sample, suggesting that the initial monoubiquitination by UBE2W was rate limiting in our in-vitro reactions. We then used the FRET-based ubiquitin discharge assays to compare the ability of the Ub-R3 WT and R3 WT samples to catalyze ubiquitin discharge from the UBE2N/V2-Ub conjugate (FIG. 4I). We observed that activity of the Ub-R3 WT sample was more than ten times higher than that of the unmodified R3 WT trimer. The FRET-based ubiquitin discharge assays are inherently single-turnover because RING constructs in these assays are present in large excess over the fluorescent UBE2N/V2-Ub conjugate. Analysis of the ubiquitin discharge products by fluorescent SDS-PAGE imaging of the AF488-labeled ubiquitin revealed that whereas the dominant product of ubiquitin discharge catalyzed by R3 WT is unconjugated di-ubiquitin, ubiquitin discharged by Ub-R3 WT is predominantly incorporated into the Ub₂-R3 WT product (FIG. 4J). Overall conclusions from the series of experiments shown in FIG. 4 are graphically summarized in the last panel of the figure (FIG. 4I).

FRET assays described herein were used to evaluate whether the proximity of three RINGs enhances the rate of inter-trimer RING autoubiquitylation compared to the rate of intermolecular reactions. To minimize the number of assay components, purified FRET-active E2˜Ub conjugates were used in this set of ubiquitin transfer reactions. The K63 residue of ubiquitin is the preferred substrate of the UBE2N˜Ub/V2 conjugate, but the N-terminal amine of ubiquitin is not a good substrate for UBE2W˜Ub because ubiquitin lacks a flexible N-terminus. To provide a good alternative substrate for both, UBE2N/V2-mediated and UBE2W-mediated ubiquitylation, we used an engineered ubiquitin, which contained a 5-residue (GGGGS (SEQ ID NO:8) N-terminal extension preceding the starting methionine of the WT ubiquitin (N5-Ub). Different RING constructs and 5 μM of the N5-Ub substrate were added to the UBE2W˜Ub or UBE2N-Ub/V2 conjugates and the FRET signal was used to monitor ubiquitin discharge from the conjugate. In addition, series of time point samples were collected for the analysis of the products by SDS-PAGE imaged using AF488 fluorescence.

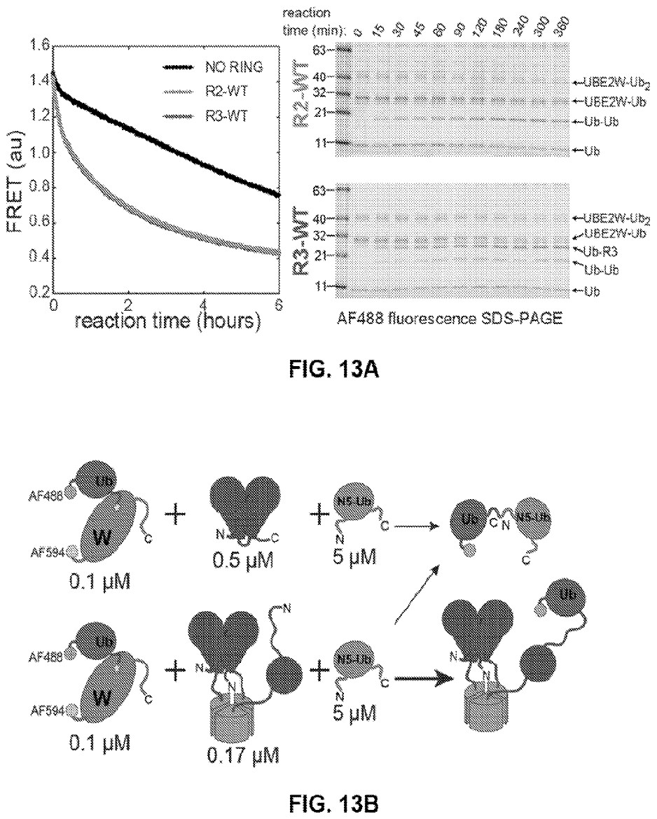

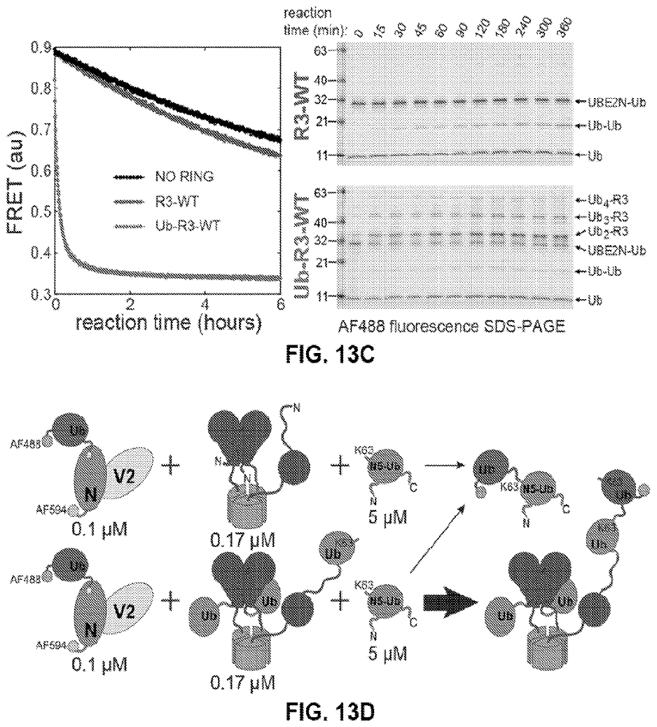

First, ubiquitin transfer from the UBE2W˜Ub conjugate catalyzed by the R2-WT and R3-WT constructs (FIG. 13A and FIG. 13B) were compared. Structures of the RING dimer complexes with E2˜Ub conjugates reveal that the same RING dimer is unlikely to act as both the catalytic activator and the substrate in ubiquitin transfer. In agreement, the main product observed in the reaction of 0.1 μM UBE2W˜Ub, 0.5 μM R2-WT and 5 μM N5-Ub mixture is di-ubiquitin (Ub-Ub) formed in an “intermolecular” transfer of AF488-Ub from UBE2W˜Ub onto the N5-Ub substrate. In contrast, in the reaction of 0.1 μM UBE2W˜Ub, 0.5 μM R3-WT and 5 M N5-Ub, the dominant product is the monoubiquitylated Ub-R3-WT despite the 30-fold excess of N5-Ub over RING trimer. These observations strongly support the “intramolecular” mechanism of R3-WT monoubiquitylation, in which the catalytic RING dimer and the substrate RING reside within the same R3-WT trimer.

How proximity of three RINGs affects the rates of ubiquitin transfer reactions mediated by UBE2N/V2 (FIG. 13C and FIG. 13D) were explored. To that end a Ub-R3-WT construct was prepared, in which the full-length WT Ub sequence is cloned immediately preceding the residue A2 of the RING, yielding the fusion protein that is chemically identical to the product of N-terminal monoubiquitylation of R3-WT by the UBE2W. Once again, the dominant product of the reaction containing 0.1 μM UBE2N-Ub/V2, 0.5 μM R3-WT and 5 M N5-Ub was di-ubiquitin (Ub-Ub) as expected for the intermolecular transfer of 488-Ub from UBE2N˜Ub onto K63 of N5-Ub. Notably, when the reaction is performed with Ub-R3-WT instead of R3-WT, the rate of FRET decay is dramatically enhanced, and the RING-conjugated products (Ub₂-R3-WT and Ubₙ-R3-WT) are greatly favored. The striking increase of the apparent ubiquitin transfer rate reveals that monoubiquitylated RING trimer is an exceptionally good substrate for ubiquitin chain extension by UBE2N-Ub/V2 owing to the proximity of the monoubiquitylated substrate RING monomer to the catalytic RING dimer in the Ub-R3-WT construct. Collectively, the data in FIG. 4 and FIG. 13 establish that proximity of three RINGs accelerates the rates of the priming and extension reactions in the autoubiquitylation of TRIM5α.

### B. Materials and Methods

Protein Expression and Purification. All recombinant proteins used in the study were expressed in bacteria and purified using standard protocols. All protein modifications (mutations, insertions and deletions) were performed using the QuickChange Lightning Multi Site-Directed Mutagenesis Kit (Agilent Cat #200513). Isotopic labeling of proteins for NMR studies was achieved by culturing bacteria in isotopically enriched minimal media (M9) following standard procedures. The R1 constructs encompass residues 2-90 of rhesus monkey TRIM5α. The R2 tandem constructs had the following residue composition: 2-82-Gly-Ser-2-92. The R3 construct contained residues 2-95 fused on the C-terminus to the T4 fibritin foldon peptide sequence followed by a 6His tag. R1 and R2 were cloned into custom pET30a expression vectors as fusions containing a 6His tag, followed by a GB1 solubility tag, followed by a TEV cleavage site at the N-terminus. The tags were removed by TEV protease digestion using standard protocols. The R3 construct was cloned into a pET11a vector. The start-codon N-formylmethionine in the expressed protein was naturally removed by the bacterial methionine aminopeptidase. Expression and purification of E2s and ubiquitin was performed using previously published procedures (Pickart and Raasi, Methods Enzymol 399, 21-36, 2005). Sortase recognition peptides or FLAG tags were added to some constructs as described.

NMR spectroscopy. All NMR experiments were performed in 50 mM Sodium Phosphate (pH 7.5), 150 mM NaCl, 1 mM TCEP, 10% (v/v) D₂O, 0.04% (w/v) NaN₃, and the data collected at 298K on a Bruker Avance 700-MHz spectrometer equipped with a cryoprobe. Backbone assignments were carried out using standard TROSY triple resonance NMR experiments (HNCO, HN(CA)CO, HN(CO)CA, HNCA, HNCACB, and HN(CO)CACB). Protein concentrations for each experiment are shown in the corresponding figures or noted in figure legends. The NMR data were processed using NMRPipe and spectra evaluated using NMRFAM-Sparky.

Analytical Ultracentrifugation. Sedimentation velocity experiments were performed with a Beckman Optima XL-I analytical ultracentrifuge (Beckman Coulter, Indianapolis, IN) equipped with a 4-hole AnTi-60 rotor and 2-channel centerpiece cells. Samples were in a buffer containing 50 mM Tris (pH 7.5), 150 mM NaCl, and 1 mM TCEP. Sedimentation velocity experiments were carried out at 20° C. and data collection performed in the intensity mode at 280 nm. Sedimentation velocity datasets were analyzed with SEDFIT 16.1 using continuous size distribution c(S) model. All c(S) distributions were calculated with fitted frictional ratios (f/f₀) and meniscus positions were all fitted with a maximum entropy regularization cutoff of 0.95. Sedimentation equilibrium experiments were carried out using six-channel centerpieces, each chamber containing 110 μL samples. Equilibrium experiments were conducted at 20° C. and collection of data performed in the intensity mode at 280 nm and 230 nm. A total of 60 scans were collected at each equilibrium speed and analyzed using SEDFIT 16.1 to determine when equilibrium was reached. Final scans for each sample and speeds were analyzed using SEDPHAT 15.2b. Equilibrium distributions were fitted to a monomer-dimer self-association model.

Sortase labeling. Fluorescent labeling of proteins was carried out by sortase-catalyzed transpeptidation as previously described⁴⁸. Briefly, for N-terminal labeling of E2s and ubiquitin the proteins were expressed with N-terminal affinity tags followed by a TEV protease cleavage site. Treatment with TEV protease was used to remove the fusion tag leaving an N-terminal glycine residue on the cleaved protein. Short fluorescent peptides used for the N-terminal sortase-catalyzed labeling contained a fluorescent dye at the N terminus and a sortase recognition sequence (KLPETGG (SEQ ID NO:6)) at the C terminus. Labeling reactions were carried out using 80 μM fluorescently labeled peptide, 20 μM sortase and 20 μM of the target protein for 30 min at 37° C. Unreacted fluorescent peptides were separated from the labeled proteins by size exclusion chromatography. C-terminal labeling of the R3-WT construct was achieved by adding a sortase recognition sequence (KLPETGG (SEQ ID NO:6)) at the C terminus of the construct and using a short fluorescent peptide containing a four glycine sequence at the N terminus and the AF594 fluorescent dye at the C terminus.

Ubiquitination Assays. Unless specified otherwise ubiquitination reactions were performed in 100 μL volumes and contained 1 mg/mL of E1, 0.1-1 μM E2, 1 μM E3, 50 μM Ub, and 5 mM ATP in the reaction buffer (50 mM Tris, pH 7.4, 150 mM NaCl, 5 mM MgCl2, 1 mM DTT, 1% (v/v) glycerol, 3 U/mL creatine phosphokinase, and 5 mM creatine phosphate). For reactions, containing fluorescent Ub, reactions were supplemented with 5 μM fluorescent Ub. Reactions were incubated at 37° C. for 1 hr. Reactions were quenched with the addition of reducing SDS-PAGE buffer and loaded onto SDS-PAGE gels (Invitrogen Cat #XP102005BOX). Following SDS-PAGE, gels were either imaged using a Typhoon TRIO imager or transferred onto nitrocellulose membranes for western blotting.

For Western immunoblotting, membranes were probed using anti-Ub (1:1,000-CST Cat #3933S), anti-K63 Ub (1:1,000-CST Cat #3496S), or anti-FLAG (1:5,000-Millipore Cat #F1804). Secondary fluorescent antibodies (1:10,000-Licor Cat #926-3211 and 926-68073) were used to detect Ub and RING products using a LI-COR Odyssey scanner. Western blot analysis was performed using ImageStudio Lite (Ver 5.2).

FRET Ubiquitin Discharge Assays. Fluorescently labeled E2˜Ub conjugates for FRET-based ubiquitin discharge assays were prepared by incubating 2.5 mg/mL E1, 5 μM AF594-labeled E2, 5.5 μM AF488-labeled Ub and 5 mM ATP in conjugation buffer (50 mM Tris pH 7, 150 mM NaCl, 5 mM MgCl₂, 1 mm DTT, 1% (v/v) glycerol, 0.5 mg/mL BSA) for 30 min at 37° C. The reactions were then quenched by depleting ATP with 2 U/mL of apyrase (New England BioLabs Cat #M0398S) for 5 min at room temperature. The reactions were then diluted and dispensed into 384-well fluorescence microplate to yield 125 nM final concentration of the fluorescent E2˜Ub conjugate. Discharge reactions were run in discharge buffer (50 mM Tris pH 7, 150 mM NaCl, 5 mM MgCl₂, 1% (v/v) glycerol, 0.5 mg/mL BSA, 10 μM free unlabeled ubiquitin) at room temperature by adding different amounts of RING constructs to the fluorescent E2˜Ub conjugates. Discharge rates were not significantly affected when the concentration of the free unlabeled ubiquitin in the discharge buffer was increased from 10 μM to 50 μM. Fluorescence intensity measurements were performed in 384-well plates on a Synergy 2 microplate reader (BioTek). All experiments were performed in duplicate with 70-μL sample volumes in each well.

Fluorescence decay curves were fit in MATLAB (R2019b) to single exponential decays as described.

1. A FRET-active E2-Ub/Ubl conjugate comprising a ubiquitin (Ub) or a ubiquitin-like protein (Ubl) covalently coupled to an E2 protein, the Ub or Ubl protein being modified with a first fluorophore and the E2 protein being modified with a second fluorophore, wherein the first fluorophore and the second fluorophore can transfer energy from one to another via Fluorescence Resonance Energy Transfer (FRET) when in proximity of each other.

2. The conjugate of claim 1, wherein the carboxyl terminus of the Ub or Ubl is covalently coupled to the E2 protein through a thioester bond with the active-site cysteine of the E2 protein.

3. The conjugate of claim 1, wherein the first fluorophore is a fluorescent dye or quencher.

4. The conjugate of claim 1, wherein the second fluorophore is a fluorescent dye or quencher.

5. The conjugate of claim 1, wherein the FRET-active E2-Ub or E2-Ubl conjugate is a fluorescent complex or a quenched complex.

6. The conjugate of claim 1, wherein the two fluorophores form a FRET pair or two fluorophores form a quenched FRET pair.

7. (canceled)

8. (canceled)

9. The conjugate of claim 8, wherein the FRET-active E2-Ub or E2-Ubl conjugate comprises a quencher.

10. The conjugate of claim 9, wherein the quencher is selected from 4-(4′-dimethylaminophenylazo)benzoic acid) (DABCYL), DABSYL, QSY-7, QSY-33, BHQ0, BHQ1, BHQ3, BHQ4, Iowa Black™ BlackBerry™ Quencher 650, cyanine dyes, anthraquinone, malachite green, nitrothiazole, nitroimidazole compounds, FAM, TET, JOE, HEX, Oregon Green®, TAMRA, ROX, Cyanine-3, Cyanine-3.5, Cyanine-5 and Cyanine-5.5.

11. The conjugate of claim 1, wherein the first fluorophore or the second fluorophore is a long fluorescence lifetime fluorophore.

12. The conjugate of claim 11, wherein the long fluorescence lifetime fluorophore is a lanthanide chelate or a lanthanide cryptate.

13. (canceled)

14. (canceled)

15. A method for monitoring activity of a E3 ubiquitin ligase comprising:
(i) contacting a FRET-active E2-Ubl conjugate of claim 1 with a sample to be monitored or assessed for a fluorescent signal or a quenched signal from an intact E2-Ubl conjugate, and
(ii) detecting dissociation of the E2-Ubl conjugate by detecting an increase in a fluorescent signal or a reduction in a fluorescent signal, the change in signal over time being produced in the presence of a polypeptide having a E3 ubiquitin ligase activity whose ligase activity results in the dissociation of the E2-Ubl conjugate.

16. (canceled)

17. (canceled)

18. (canceled)

19. (canceled)

20. (canceled)

21. (canceled)

22. (canceled)

23. (canceled)

24. (canceled)

25. (canceled)

26. A method for monitoring activity of a E1 ubiquitin activating enzyme, the method comprising:
(i) contacting a E2 protein labeled with a first fluorescent or quenching molecule and a Ubl protein labeled with a second fluorescent or quenching molecule with a sample, and
(ii) detecting a fluorescent signal or a reduction in fluorescent signal produced by conjugation of the E2 protein and the Ubl protein wherein the signal will change over time in the presence of a polypeptide having a E1 ubiquitin activating activity, ubiquitin activating activity resulting in the conjugation of the E2 protein and Ubl or Ubl-like protein and formation of a FRET-active E2-Ubl conjugate wherein the first fluorescent or quenching molecule and the second fluorescent or quenching molecule come into proximity.

27. The method of claim 26, wherein the E2 protein is the E2 protein of claim 13.

28. The method of claim 26, wherein the Ubl or Ubl-like protein is labeled with a first fluorophore and the E2 protein is labeled with a second fluorophore, the first fluorophore and second fluorophore forming a fluorescent resonance energy transfer (FRET) pair.

29. The method of claim 26, wherein the FRET pair is selected from Alexa Fluor® 350, Alexa Fluor® 488, Alexa 25 Fluor® 532, Alexa Fluor® 546, Alexa Fluor® 568, Alexa Fluor® 594, or Alexa Fluor® 647, 4-(4′-dimethylaminophenylazo)benzoic acid) (DABCYL), DABSYL, QSY-7, QSY-33, BHQ0, BHQ1, BHQ3, BHQ4, Iowa Black™ BlackBerry™ Quencher 650, cyanine dyes, anthraquinone, malachite green, nitrothiazole, nitroimidazole compounds, FAM, TET, JOE, HEX, Oregon Green®, TAMRA, ROX, Cyanine-3, Cyanine-3.5, Cyanine-5 and Cyanine-5.5.

30. The method of claim 26, wherein the FRET pair comprises an Alexa Fluor 488 and an Alexa Fluor 594.

31. The method of claim 26, wherein the E2 protein is selected from a protein encoded by E2 ubiquitin conjugating enzyme gene UBE2A, UBE2B, UBE2C, UBE2D1, UBE2D2, UBE2D3, UBE2E1, UBE2E2, UBE2E3, UBE2F, UBE2G1, UBE2G2, UBE2H, UBE2I, UBE2J1, UBE2J2, UBE2K, UBE2L3, UBE2L6, UBE2M, UBE2N, UBE20, UBE2Q1, UBE2Q2, UBE2R1, UBE2R2, UBE2S, UBE2T, UBE2U, UBE2V1, UBE2V2, UBE2Z, ATG2, BIRC5, or UFC.

32. (canceled)

33. The method of claim 26, wherein the Ubl protein is selected from a protein encoded by Ubl gene SUMO1, SUMO2, SUMO3, SUMO4, NEDD8, ATG8, ATG12, URM1, UFM1, FAT10, and ISG15.

34. (canceled)

35. (canceled)

36. A kit for synthesis of FRET-active E2-Ub/Ubl conjugates comprising a Ub/Ubl labeled with a first fluorophore, an E2 labeled with a second fluorophore, such that the first and the second fluorophores make a FRET-active pair.

37. (canceled)

38. (canceled)


SEQUENCE LISTING (16 sequences)

SEQ ID NO: 1 - protein, 76 aa, Homo sapiens [sequence omitted]

SEQ ID NO: 2 - protein, 229 aa, Homo sapiens
MQIFVKTLTGKTITLEVEPSDTIENVKAKIQDKEGIPPDQQRLIFAGKQLEDGRTLSDYNIQKESTLHLVLRLRGGMQIFVKTLTGKTITLEVEPSDTIENVKAKIQDKEGIPPDQQRLIFAGKQLEDGRTLSDYNIQKESTLHLVLRLRGGMQIFVKTLTGKTITLEVEPSDTIENVKAKIQDKEGIPPDQQRLIFAGKQLEDGRTLSDYNIQKESTLHLVLRLRGGC

SEQ ID NO: 3 - protein, 1058 aa, Homo sapiens
MSSSPLSKKRRVSGPDPKPGSNCSPAQSVLSEVPSVPTNGMAKNGSEADIDEGLYSRQLYVLGHEAMKRLQTSSVLVSGLRGLGVEIAKNIILGGVKAVTLHDQGTAQWADLSSQFYLREEDIGKNRAEVSQPRLAELNSYVPVTAYTGPLVEDFLSGFQVVVLTNTPLEDQLRVGEFCHNRGIKLVVAGTRGLFGQLFCDFGEEMILTDSNGEQPLSAMVSMVTKDNPGVVTCLDEARHGFESGDFVSFSEVQGMVELNGNQPMEIKVLGPYTFSICDTSNFSDYIRGGIVSQVKVPKKISFKSLVASLAEPDFVVTDFAKFSRPAQLHIGFQALHQFCAQHGRPPRPRNEEDAAELVALAQAVNARALPAVQQNNLDEDLIRKLAYVAAGDLAPINAFIGGLAAQEVMKACSGKFMPIMQWLYFDALECLPQDKEVLTEDKCLQRQNRYDGQVAVFGSDLQEKLGKQKYFLVGAGAIGCELLKNFAMIGLGCGEGGEIIVTDMDTIEKSNLNRQFLFRPWDVTKLKSDTAAAAVRQMNPHIRVTSHQNRVGPDTERIYDDDFFQNLDGVANALDNVDARMYMDRRCVYYRKPLLESGTLGTKGNVQVVIPFLTESYSSSQDPPEKSIPICTLKNFPNAIEHTLQWARDEFEGLFKQPAENVNQYLTDPKFVERTLRLAGTQPLEVLEAVQRSLVLQRPQTWADCVTWACHHWHTQYSNNIRQLLHNFPPDQLTSSGAPFWSGPKRCPHPLTFDVNNPLHLDYVMAAANLFAQTYGLTGSQDRAAVATFLQSVQVPEFTPKSGVKIHVSDQELQSANASVDDSRLEELKATLPSPDKLPGFKMYPIDFEKDDDSNFHMDFIVAASNLRAENYDIPSADRHKSKLIAGKIIPAIATTTAAVVGLVCLELYKVVQGHRQLDSYKNGFLNLALPFFGFSEPLAAPRHQYYNQEWTLWDRFEVQGLQPNGEEMTLKQFLDYFKTEHKLEITMLSQGVSMLYSFFMPAAKLKERLDQPMTEIVSRVSKRKLGRHVRALVLELCCNDESGEDVEVPYVRYTIR

SEQ ID NO: 4 - protein, 147 aa, Homo sapiens
MALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAM

SEQ ID NO: 5 - protein, 493 aa, Homo sapiens
MASGILVNVKEEVTCPICLELLTQPLSLDCGHSFCQACLTANHKKSMLDKGESSCPVCRISYQPENIRPNRHVANIVEKLREVKLSPEGQKVDHCARHGEKLLLFCQEDGKVICWLCERSQEHRGHHTFLTEEVAREYQVKLQAALEMLRQKQQEAEELEADIREEKASWKTQIQYDKTNVLADFEQLRDILDWEESNELQNLEKEEEDILKSLTNSETEMVQQTQSLRELISDLEHRLQGSVMELLQGVDGVIKRTENVTLKKPETFPKNQRRVFRAPDLKGMLEVFRELTDVRRYWVDVTVAPNNISCAVISEDKRQVSSPKPQIIYGARGTRYQTFVNFNYCTGILGSQSITSGKHYWEVDVSKKTAWILGVCAGFQPDAMCNIEKNENYQPKYGYWVIGLEEGVKCSAFQDSSFHTPSVPFIVPLSVIICPDRVGVFLDYEACTVSFFNITNHGFLIYKFSHCSFSQPVFPYLNPRKCGVPMTLCSPSS

SEQ ID NO: 6 - protein, 7 aa, synthetic construct
  REGION 1..7: note = synthetic construct
KLPETGG

SEQ ID NO: 7 - protein, 5 aa, synthetic construct
  REGION 1..5: note = synthetic construct
  VARIANT 3: note = X is any amino acid
LPXTG

SEQ ID NO: 8 - protein, 5 aa, synthetic construct
  REGION 1..5: note = synthetic consrtuct
GGGGS

SEQ ID NO: 9 - protein, 12 aa, synthetic construct
RHVANIVEKLRE

SEQ ID NO: 10 - protein, 20 aa, synthetic construct
KLSPEEGQKVDGYIPEAPRD

SEQ ID NO: 11 - protein, 21 aa, synthetic construct
RKDGEWVLLSTFLSGHHHHHH

SEQ ID NO: 12 - protein, 13 aa, synthetic construct
REVKLSPEEGQKV

SEQ ID NO: 13 - protein, 15 aa, synthetic construct
KLREVKLSPEEGQKV

SEQ ID NO: 14 - protein, 11 aa, synthetic construct
MASGILLNVKE

SEQ ID NO: 15 - protein, 11 aa, synthetic construct
KSMLYKEGERS

SEQ ID NO: 16 - protein, 11 aa, synthetic construct
GGASGILLNVK